SEQUENCE LISTING (33 sequences)

SEQ ID NO: 1 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 2 - DNA, 7570 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatctcaccatggagttgggactgcgctggggcttcctcgttgctcttttaagaggtgtccagtgtcaggtgcaattggtggagtctgggggaggcgtggtccagcctgggaggtccctgagactctcctgtgcagcgtctggattcgccttcagtagatatggcatgcactgggtccgccaggctccaggcaaggggctggagtgggtggcagttatatggtatgatggaagtaataaatactatgcagactccgtgaagggccgattcaccatctccagagacaattccaagaacacgcagtatctgcaaatgaacagcctgagagccgaggacacggctgtgtattactgtgcgagaggcggtgacttcctctactactactattacggtatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 7597 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatctcaccatggggtcaaccgccatcctcaccatggagttggggctgcgctgggttctcctcgttgctcttttaagaggtgtccagtgtcaggtgcagctggtggagtctgggggaggcgtggtccagcctgggaggtccctgagactctcctgtgcagcgtctggattcaccttcagtaactatgtcatgcactgggtccgccaggctccaggcaaggggctggagtgggtggcaattatatggtatgatggaagtaataaatactatgcagactccgtgaagggccgattcaccatctccagagacaattccaagaacacgctgtatctgcaaatgaacagcctgagagccgaggacacggctgtgtattactgtgcgggtggatataactggaactacgagtaccactactacggtatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 7579 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatctcaccatggagttgggacttagctgggttttcctcgttgctcttttaagaggtgtccagtgtcaggtccagctggtggagtctgggggaggcgtggtccagcctgggaggtccctgagactctcctgtgcagcgtctggattcaccttcagtagctatggcatgcactgggtccgccaggctccaggcaaggggctggactgggtggcaattatttggcatgatggaagtaataaatactatgcagactccgtgaagggccgattcaccatctccagagacaattccaagaagacgctgtacctgcaaatgaacagtttgagagccgaggacacggctgtgtattactgtgcgagagcttgggcctatgactacggtgactatgaatactacttcggtatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 7558 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatcccaccatggggtcaaccgtcatcctcgccctcctcctggctgttctccaaggagtctgtgccgaggtgcagctggtgcagtctggagcagaggtgaaaaagcccggggagtctctgaagatctcctgtaagggttctggatacagctttaccagttactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacggatggcagcagctggcccctttgactactggggccagggaaccctggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 7576 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatctcaccatggagtttgggctgtgctggattttcctcgttgctcttttaagaggtgtccagtgtcaggtgcagctggtggagtctgggggaggcgtggtccagcctgggaggtccctgagactctcctgtgcagcctctggattcaccttcattagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgagagtattagtgggagctttatattattataactactacgggatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - DNA, 7561 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccggatctcaccatggagttggggctgagctgggttttcctcgttgctcttttaagaggtgtccagtgtcaggagcagctggtggagtctgggggaggcgtggtccagcctgggaggtccctgagactctcctgtgcagcgtctggattcaccttcagtacctatggcatgcactgggtccgccaggctccaggcaaggggctggagtgggtggcagttacatggcatgatggaagtaataaatactatgcagactccgtgaagggccgattcaccatctccagagacaactccaagaacacgctgtatctgcaaatgaacagcctgagagccgaggacacggctgtgtattactgtgcgagaggaggagtgggagcaacttactactactactacggtatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - DNA, 6082 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccctgctcagctcctgggactcctgctgctctggctcccagataccagatgtgacatccagatgacccagtctccatcctccctgtctgcatctgtaggagacagagtcaccatcacttgccgggcgagtcagggcattagcaattatttagcctggtatcagcagaaaacagggaaagttcctaagttcctgatctatgaagcatccactttgcaatcaggggtcccatctcggttcagtggcggtggatctgggacagatttcactctcaccatcagcagcctgcagcctgaagatgttgcaacttattactgtcaaaattataacagtgccccattcactttcggccctgggaccaaagtggatatcaaacgaactgtggctgcaccctctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - DNA, 6082 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccccgctcagctcctggggctcctgctgctctgtttcccaggtgccagatgtgacatccagatgacccagtctccatcctcactgtctgcatctgtaggagacagagtcaccatcacttgtcgggcgagtcagggcattaccaattatttagcctggtttcagcagaaaccagggaaagcccctaagtcccttatctatgctgcatccagtttgcaaagtggggtcccatcaaagttcagcggcagtggatctgggacagatttcagtctcaccatcagcagcctgcagcctgaagattttgcaacttattactgccaacagtataatagttacccgatcaccttcggccaagggacacgactggagattaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 10 - DNA, 6082 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccctgctcagctcctggggctcctgctgctctgtttcccaggtgccagatgtgacatccagatgacccagtctccatcctcactgtctgcatctgtaggagacagagtcaccatcacttgtcgggcgagtcagggcattagccattatttagcctggtttcagcagaaaccagggaaagcccctaagtccctgatctatgctgcatccagtttgcaaagtggggtcccatcaaagttcagcggcagtggatctgggacagatttcactctcaccatcagcagcctacagcctgaagattttgcaacttattactgccaacagtataatagtttcccgctcactttcggcggagggaccaaggtggagatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 11 - DNA, 6085 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccccgctcagcttctcttccttctgctactctggctcccagataccactggaggaatagtgatgacgcagtctccagccaccctgtctgtgtctccaggggaaagagccaccctctcctgcaggaccagtcagagtattggctggaacttagcctggtaccaacagaaacctggccaggctcccaggctcctcatctatggtgcatcttccaggaccactggtatcccagccaggttcagtggcagtgggtctgggacagagttcactctcaccatcagcagcctgcagtctgaagattctgcagtttattactgtcagcattatgataactggcccatgtgcagttttggccaggggaccgagctggagatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 12 - DNA, 6097 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccctgctcagctcctggggctgctaatgctctggatacctggatccagtgcagatattgtgatgacccagactccactctctctgtccgtcacccctggacagccggcctccatctcctgcaagtctagtcagagcctcctgcatagtgatggaaagacctttttgtattggtatctgcagaagccaggccagcctccacagctcctgatctatgaggtttccaaccggttctctggagtgccagataggttcagtggcagcgggtcagggacagatttcacactgaaaatcagccgggtggaggctgaggatgttgggctttattactgcatgcaaagtatacagcttccgctcactttcggcggagggaccaaggtggagatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 13 - DNA, 6094 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatggtgttgcagacccaggtcttcatttctctgttactctggatctctggtgcctacggggacatcgtgatgacccagtctccagactccctggctgtgtctctgggcgagagggccaccatcaactgcaagtccaaccagagtgtcttacacagctccaacaataagaactatttagcttggtaccagcagaaaccaggacagcctcctaaattgctcatttattgggcattcctccgggaatccggggtccctgaccgcttcagtggcagcgggtctgggacagatttcactctcaccatcagcagcctgcaggctgaagatgtggcagtttattactgtcaccaatattattctactttatatactttcggcggagggaccaaggtagagatcaaacgaacygtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - DNA, 481 bp, Artificial Sequence
  Includes BamHI/Bg1II cloning junction, signal peptide, V region, portion of C region and 3′XbaI/NheI (heavy) or NheI (light) cloning junction
ggatctcaccatggagttgggactgcgctggggcttcctcgttgctcttttaagaggtgtccagtgtcaggtgcaattggtggagtctgggggaggcgtggtccagcctgggaggtccctgagactctcctgtgcagcgtctggattcgccttcagtagatatggcatgcactgggtccgccaggctccaggcaaggggctggagtgggtggcagttatatggtatgatggaagtaataaatactatgcagactccgtgaagggccgattcaccatctccagagacaattccaagaacacgcagtatctgcaaatgaacagcctgagagccgaggacacggctgtgtattactgtgcgagaggcggtgacttcctctactactactattacggtatggacgtctggggccaagggaccacggtcaccgtctcctcagcctccaccaagggcccatcggtcttccccctggcaccctctagc

SEQ ID NO: 15 - protein, 142 aa, Homo sapiens
MELGLRWGFLVALLRGVQCQVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSS

SEQ ID NO: 16 - DNA, 463 bp, Artificial Sequence
  Includes BamHI/Bg1II cloning junction, signal peptide, V region, portion of C region and 3′XbaI/NheI (heavy) or NheI (light) cloning junction
ggatctcaccatgagggtccctgctcagctcctgggactcctgctgctctggctcccagataccagatgtgacatccagatgacccagtctccatcctccctgtctgcatctgtaggagacagagtcaccatcacttgccgggcgagtcagggcattagcaattatttagcctggtatcagcagaaaacagggaaagttcctaagttcctgatctatgaagcatccactttgcaatcaggggtcccatctcggttcagtggcggtggatctgggacagatttcactctcaccatcagcagcctgcagcctgaagatgttgcaacttattactgtcaaaattataacagtgccccattcactttcggccctgggaccaaagtggatatcaaacgaactgtggctgcaccctctgtcttcatcttcccgccatctgatgagcagttgaaatctggaactgctagc

SEQ ID NO: 17 - protein, 127 aa, Homo sapiens
MRVPAQLLGLLLLWLPDTRCDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIK

SEQ ID NO: 18 - DNA, 508 bp, Artificial Sequence
  Includes BamHI/Bg1II cloning junction, signal peptide, V region, portion of C region and 3′XbaI/NheI (heavy) or NheI (light) cloning junction
ggatctcaccatggggtcaaccgccatcctcaccatggagttggggctgcgctgggttctcctcgttgctcttttaagaggtgtccagtgtcaggtgcagctggtggagtctgggggaggcgtggtccagcctgggaggtccctgagactctcctgtgcagcgtctggattcaccttcagtaactatgtcatgcactgggtccgccaggctccaggcaaggggctggagtgggtggcaattatatggtatgatggaagtaataaatactatgcagactccgtgaagggccgattcaccatctccagagacaattccaagaacacgctgtatctgcaaatgaacagcctgagagccgaggacacggctgtgtattactgtgcgggtggatataactggaactacgagtaccactactacggtatggacgtctggggccaagggaccacggtcaccgtctcctcagcctccaccaagggcccatcggtcttccccctggcaccctctagc

SEQ ID NO: 19 - protein, 143 aa, Homo sapiens
MELGLRWVLLVALLRGVQCQVQLVESGGGVVQPGRSLRLSCAASGFTFSNYVMHWVRQAPGKGLEWVAIIWYDGSNKYYADSVKGRFTISRDNSKNTLYLQMNSLRAEDTAVYYCAGGYNWNYEYHYYGMDVWGQGTTVTVSS

SEQ ID NO: 20 - DNA, 463 bp, Artificial Sequence
  Includes BamHI/Bg1II cloning junction, signal peptide, V region, portion of C region and 3′XbaI/NheI (heavy) or NheI (light) cloning junction
ggatctcaccatgagggtccccgctcagctcctggggctcctgctgctctgtttcccaggtgccagatgtgacatccagatgacccagtctccatcctcactgtctgcatctgtaggagacagagtcaccatcacttgtcgggcgagtcagggcattaccaattatttagcctggtttcagcagaaaccagggaaagcccctaagtcccttatctatgctgcatccagtttgcaaagtggggtcccatcaaagttcagcggcagtggatctgggacagatttcagtctcaccatcagcagcctgcagcctgaagattttgcaacttattactgccaacagtataatagttacccgatcaccttcggccaagggacacgactggagattaaacgaactgtggctgcaccatctgtcttcatcttcccgccatctgatgagcagttgaaatctggaactgctagc

SEQ ID NO: 21 - protein, 127 aa, Homo sapiens
MRVPAQLLGLLLLCFPGARCDIQMTQSPSSLSASVGDRVTITCRASQGITNYLAWFQQKPGKAPKSLIYAASSLQSGVPSKFSGSGSGTDFSLTISSLQPEDFATYYCQQYNSYPITFGQGTRLEIK

SEQ ID NO: 22 - DNA, 490 bp, Artificial Sequence
  Includes BamHI/Bg1II cloning junction, signal peptide, V region, portion of C region and 3′XbaI/NheI (heavy) or NheI (light) cloning junction
ggatctcaccatggagttgggacttagctgggttttcctcgttgctcttttaagaggtgtccagtgtcaggtccagctggtggagtctgggggaggcgtggtccagcctgggaggtccctgagactctcctgtgcagcgtctggattcaccttcagtagctatggcatgcactgggtccgccaggctccaggcaaggggctggactgggtggcaattatttggcatgatggaagtaataaatactatgcagactccgtgaagggccgattcaccatctccagagacaattccaagaagacgctgtacctgcaaatgaacagtttgagagccgaggacacggctgtgtattactgtgcgagagcttgggcctatgactacggtgactatgaatactacttcggtatggacgtctggggccaagggaccacggtcaccgtctcctcagcctccaccaagggcccatcggtcttccccctggcaccctctagc

SEQ ID NO: 23 - protein, 145 aa, Homo sapiens
MELGLSWVFLVALLRGVQCQVQLVESGGGVVQPGRSLRLSCAASGFTFSSYGMHWVRQAPGKGLDWVAIIWHDGSNKYYADSVKGRFTISRDNSKKTLYLQMNSLRAEDTAVYYCARAWAYDYGDYEYYFGMDVWGQGTTVTVSS

SEQ ID NO: 24 - DNA, 463 bp, Artificial Sequence
  Includes BamHI/Bg1II cloning junction, signal peptide, V region, portion of C region and 3′XbaI/NheI (heavy) or NheI (light) cloning junction
ggatctcaccatgagggtccctgctcagctcctggggctcctgctgctctgtttcccaggtgccagatgtgacatccagatgacccagtctccatcctcactgtctgcatctgtaggagacagagtcaccatcacttgtcgggcgagtcagggcattagccattatttagcctggtttcagcagaaaccagggaaagcccctaagtccctgatctatgctgcatccagtttgcaaagtggggtcccatcaaagttcagcggcagtggatctgggacagatttcactctcaccatcagcagcctacagcctgaagattttgcaacttattactgccaacagtataatagtttcccgctcactttcggcggagggaccaaggtggagatcaaacgaactgtggctgcaccatctgtcttcatcttcccgccatctgatgagcagttgaaatctggaactgctagc

SEQ ID NO: 25 - protein, 127 aa, Homo sapiens
MRVPAQLLGLLLLCFPGARCDIQMTQSPSSLSASVGDRVTITCRASQGISHYLAWFQQKPGKAPKSLIYAASSLQSGVPSKFSGSGSGTDFTLTISSLQPEDFATYYCQQYNSFPLTFGGGTKVEIK

SEQ ID NO: 26 - DNA, 469 bp, Artificial Sequence
  Includes BamHI/Bg1II cloning junction, signal peptide, V region, portion of C region and 3′XbaI/NheI (heavy) or NheI (light) cloning junction
ggatcccaccatggggtcaaccgtcatcctcgccctcctcctggctgttctccaaggagtctgtgccgaggtgcagctggtgcagtctggagcagaggtgaaaaagcccggggagtctctgaagatctcctgtaagggttctggatacagctttaccagttactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacggatggcagcagctggcccctttgactactggggccagggaaccctggtcaccgtctcctcagcctccaccaagggcccatcggtcttccccctggcaccctctagc

SEQ ID NO: 27 - protein, 138 aa, Homo sapiens
MGSTVILALLLAVLQGVCAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARRMAAAGPFDYWGQGTLVTVSS

SEQ ID NO: 28 - DNA, 466 bp, Artificial Sequence
  Includes BamHI/Bg1II cloning junction, signal peptide, V region, portion of C region and 3′XbaI/NheI (heavy) or NheI (light) cloning junction
ggatctcaccatgagggtccccgctcagcttctcttccttctgctactctggctcccagataccactggaggaatagtgatgacgcagtctccagccaccctgtctgtgtctccaggggaaagagccaccctctcctgcaggaccagtcagagtattggctggaacttagcctggtaccaacagaaacctggccaggctcccaggctcctcatctatggtgcatcttccaggaccactggtatcccagccaggttcagtggcagtgggtctgggacagagttcactctcaccatcagcagcctgcagtctgaagattctgcagtttattactgtcagcattatgataactggcccatgtgcagttttggccaggggaccgagctggagatcaaacgaactgtggctgcaccatctgtcttcatcttcccgccatctgatgagcagttgaaatctggaactgctagc

SEQ ID NO: 29 - protein, 128 aa, Homo sapiens
MRVPAQLLFLLLLWLPDTTGGIVMTQSPATLSVSPGERATLSCRTSQSIGWNLAWYQQKPGQAPRLLIYGASSRTTGIPARFSGSGSGTEFTLTISSLQSEDSAVYYCQHYDNWPMCSFGQGTELEIK

SEQ ID NO: 30 - DNA, 487 bp, Artificial Sequence
  Includes BamHI/Bg1II cloning junction, signal peptide, V region, portion of C region and 3′XbaI/NheI (heavy) or NheI (light) cloning junction
ggatctcaccatggagtttgggctgtgctggattttcctcgttgctcttttaagaggtgtccagtgtcaggtgcagctggtggagtctgggggaggcgtggtccagcctgggaggtccctgagactctcctgtgcagcctctggattcaccttcattagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgagagtattagtgggagctttatattattataactactacgggatggacgtctggggccaagggaccacggtcaccgtctcctcagcctccaccaagggcccatcggtcttccccctggcaccctctagc

SEQ ID NO: 31 - protein, 144 aa, Homo sapiens
MEFGLCWIFLVALLRGVQCQVQLVESGGGVVQPGRSLRLSCAASGFTFISYGMHWVRQAPGKGLEWVAVISYDGSNKYYADSVKGRFTISRDNSKNTLYLQMNSLRAEDTAVYYCARVLVGALYYYNYYGMDVWGQGTTVTVSS

SEQ ID NO: 32 - DNA, 478 bp, Artificial Sequence
  Includes BamHI/Bg1II cloning junction, signal peptide, V region, portion of C region and 3′XbaI/NheI (heavy) or NheI (light) cloning junction
ggatctcaccatgagggtccctgctcagctcctggggctgctaatgctctggatacctggatccagtgcagatattgtgatgacccagactccactctctctgtccgtcacccctggacagccggcctccatctcctgcaagtctagtcagagcctcctgcatagtgatggaaagacctttttgtattggtatctgcagaagccaggccagcctccacagctcctgatctatgaggtttccaaccggttctctggagtgccagataggttcagtggcagcgggtcagggacagatttcacactgaaaatcagccgggtggaggctgaggatgttgggctttattactgcatgcaaagtatacagcttccgctcactttcggcggagggaccaaggtggagatcaaacgaactgtggctgcaccatctgtcttcatcttcccgccatctgatgagcagttgaaatctggaactgctagc

SEQ ID NO: 33 - protein, 132 aa, Homo sapiens
MRVPAQLLGLLMLWIPGSSADIVMTQTPLSLSVTPGQPASISCKSSQSLLHSDGKTFLYWYLQKPGQPPQLLIYEVSNRFSGVPDRFSGSGSGTDFTLKISRVEAEDVGLYYCMQSIQLPLTFGGGTKVEIK

(19) United States
(12) Patent Application Publication
(10) Pub. No.: US 2015/0110814 A1
(43) Pub. Date: Apr. 23, 2015

(54) COMBINATION THERAPIES WITH PSMA LIGAND CONJUGATES

(71) Applicant: PSMA Development Company, LLC, Tarrytown, NY (US)

(72) Inventors: William C. Olson, Yorktown Heights, NY (US); Vincent DiPippo, Mahopac, NY (US)

(73) Assignee: PSMA Development Company, LLC, Tarrytown, NY (US)

(21) Appl. No.: 14/277,367

(22) Filed: May 14, 2014

Related U.S. Application Data
(60) Provisional application No. 61/903,589, filed on Nov. 13, 2013.
(60) Provisional application No. 61/893,145, filed on Oct. 18, 2013.

(30) Foreign Application Priority Data
Nov. 13, 2013 (JP) 2013-235506

(51) Int. Cl. A61K 38/05; A61K 31/4166; A61J 1/05; A61K 31/436; A61K 31/573; A61K 47/48; A61K 31/58

(52) U.S. Cl. CPC A61K 38/05; A61K 47/48646; A61K 31/4166; A61K 31/58; A61K 31/436; A61K 31/573; A61J 1/05
USPC 424/178.1; 435/375; 206/438

(57) ABSTRACT

Compositions and methods related to inhibiting the proliferation of or killing of prostate-specific membrane antigen (PSMA)-expressing cells are provided herein. In some embodiments, PSMA-expressing cells are contacted with (i) a compound that increases cell surface expression of PSMA and (ii) a PSMA ligand conjugate. In other embodiments, the PSMA-expressing cells are contacted with (i) prednisone and (ii) a PSMA ligand conjugate. In some of these embodiment ts, the PSMA-expressing cells are further contacted with (iii) a compound that increases cell surface expression of PSMA.

### RELATED APPLICATIONS

This application claims the benefit under 35 U.S.C. §119 of U.S. provisional application 61/893145, filed Oct. 18, 2013 and 61/903589, filed Nov. 13, 2013, the entire contents of each of which are incorporated herein by reference. Foreign priority benefits are claimed under 35 U.S.C. §119(a)-(d) or 35 U.S.C. §365(b) to Japanese application number 2013-235506, filed Nov. 13, 2013, the entire contents of which are also incorporated by reference herein.

### BACKGROUND

Prostate cancer is the most common malignancy and the second leading cause of cancer death in men in the United States (Jemal A, et al., CA Cancer J Clin 2005; 55:10-30). Localized prostate cancer typically is treated with surgery or radiation, and recurrent disease can be controlled temporarily with androgen ablation (Klein E A, et al., Urol Clin North Am 2003; 30:315-30). However, almost all prostate carcinomas eventually become hormone refractory and then rapidly progress (Denmeade S R, et al., Nat Rev Cancer 2002; 2:389-96). Hormone-refractory or androgen-independent prostate cancer has proven to be largely resistant to conventional chemotherapy. Therapies that have been shown to prolong life in subjects with metastatic, castration-resistant prostate cancer (mCRPC) include: Taxotere® (docetaxel), Jevtana® (cabazitaxel), Zytiga® (abiraterone acetate), Provenge® (sipuleucel-T), Xtandi® (enzalutamide) and Xofigo® (radium Ra-223 dichloride). The survival benefits may be modest as in the case of Jevtana®, which offer 2.4 month survival benefit (Gulley J, et al., Am J Ther. 2004; 351:1513-20; Petrylak D P, et al., New Engl J Med 2004; 351:1513-20). New therapies are needed to expand therapeutic options, such as for subjects with mCRPC.

### SUMMARY OF THE INVENTION

The present invention relates, at least in part, to the surprising discovery that antiandrogens and prostate-specific membrane antigen (PSMA) ligands bound to therapeutic agents (referred to herein as “PSMA ligand conjugates”), such as anticancer agents or cytotoxic agents (referred to herein as, when bound to a PSMA ligand, “PSMA ligand-anticancer agent conjugates” or “PSMA ligand-cytotoxic agent conjugates”, respectively), can act synergistically to inhibit proliferation of PSMA-expressing cancer cells. Unexpectedly, this synergism occurs in both androgen-dependent and androgen-independent cells. It was also discovered that mTOR inhibitors and PSMA ligand conjugates also act synergistically to inhibit proliferation of PSMA-expressing cancer cells, such as androgen-independent cells in particular. It was also discovered that prednisone and PSMA ligand conjugates also acted synergistically in combination. Thus, provided herein are methods, compositions and kits for inhibiting proliferation of, or killing, PSMA-expressing cells (e.g., cancer cells such as prostate cancer cells) as well as for sensitizing or resensitizing cells that can express PSMA, such as androgen-independent PSMA-expressing cells, to androgen therapy.

Some aspects of the disclosure provide methods of inhibiting proliferation of prostate-specific membrane antigen (PSMA)-expressing cancer cells. The methods may comprise contacting PSMA-expressing cancer cells with a compound that increases cell surface expression of PSMA, and contacting the PSMA-expressing cancer cells with a PSMA ligand-anticancer agent conjugate.

In some embodiments, the compound that increases cell surface expression of PSMA is an antiandrogen.

In some embodiments, the antiandrogen blocks enzyme cytochrome CYP17. In some embodiments, the antiandrogen blocks enzyme cytochrome CYP17A1.

In some embodiments, the antiandrogen is an androgen receptor antagonist.

In some embodiments, the antiandrogen is abiraterone, enzalutamide, nilutamide, flutamide, bicalutamide, ARN 509, galeterone or orteronel. In some embodiments, the antiandrogen is enzalutamide or abiraterone.

In some embodiments, the antiandrogen is in an amount effective to upregulate PSMA expression.

In some embodiments, the step of contacting the PSMA-expressing cancer cells with the compound that increases cell surface expression of PSMA, such as an antiandrogen, and the step of contacting the PSMA-expressing cancer cells with the PSMA ligand-anticancer agent conjugate are concurrent. In other embodiments, the step of contacting the PSMA-expressing cancer cells with the compound, such as an antiandrogen, and the step of contacting the PSMA-expressing cancer cells with the PSMA ligand-anticancer agent conjugate are sequential.

In some embodiments, the step of contacting the PSMA-expressing cancer cells with the compound, such as an antiandrogen, is prior to the step of contacting the PSMA-expressing cancer cells with the PSMA ligand-anticancer agent conjugate.

In some embodiments, the step of contacting the PSMA-expressing cancer cells with the PSMA ligand-anticancer agent conjugate occurs within one week of the step of contacting the PSMA-expressing cancer cells with the compound, such as an antiandrogen.

In some embodiments, the PSMA-expressing cancer cells are in contact with the compound, such as an antiandrogen, for at least 3 days. In other embodiments, the PSMA-expressing cancer cells are in contact with the compound, such as an antiandrogen, for at least 7 days. In yet other embodiments, the PSMA-expressing cancer cells are in contact with the compound, such as an antiandrogen, for at least 14 days. In still other embodiments, the PSMA-expressing cancer cells are in contact with the compound, such as an antiandrogen, for at least 21 days. In still other embodiments, the PSMA-expressing cancer cells are in contact with the compound, such as an antiandrogen, for at least 28 days.

In some embodiments, the PSMA-expressing cancer cells comprise androgen-independent PSMA-expressing cancer cells.

In some embodiments, the PSMA-expressing cancer cells comprise PSMA-expressing cancer cells insensitive to antiandrogen therapy.

In some embodiments, the PSMA-expressing cancer cells insensitive to antiandrogen therapy are re-sensitized to antiandrogen therapy after the step of contacting the PSMA-expressing cancer cells with the compound, such as an antiandrogen.

In some embodiments, the PSMA-expressing cancer cells are of a tumor.

In some embodiments, the PSMA-expressing cancer cells are PSMA-expressing prostate cancer cells. In other embodiments, the PSMA-expressing cancer cells are PSMA-expressing non-prostate cancer cells. In yet other embodiments, the PSMA-expressing cancer cells are of the neovasculature of a non-prostate cancer or non-prostate tumor.

In some embodiments, the PSMA-expressing cancer cells are of a subject. In some embodiments, the subject has progressive metastatic castration-resistant prostate cancer. In some embodiments, the subject has had prior chemotherapy with one or more taxanes. In some embodiments, the subject has had prior treatment with one or more antiandrogens. In some embodiments, the subject has prostate cancer that has progressed despite prior treatment, such as with one or more antiandrogens. In some embodiments, the subject has prostate cancer that is starting to progress despite treatment, such as with one or more antiandrogens. In some embodiments, the subject has not had prior cytotoxic chemotherapy. In some embodiments, the subject has not had prior treatment with one or more antiandrogens. In some embodiments, the subject has not had prior treatment with enzalutamide or abiraterone. In some embodiments, the subject that has not had prior treatment with antiandrogens, such as enzalutamide or abiraterone, is one with metastatic castration-resistant prostate cancer. In some embodiments, the subject is any one of the subjects described herein.

Other aspects of the disclosure provide methods of inhibiting proliferation of prostate-specific membrane antigen (PSMA)-expressing cancer cells. The methods may comprise contacting PSMA-expressing cancer cells with an mTOR inhibitor, and contacting the PSMA-expressing cancer cells with a PSMA ligand-anticancer agent conjugate.

In some embodiments, the mTOR inhibitor is rapamycin.

In some embodiments, the mTOR inhibitor is in an amount effective to upregulate PSMA expression.

In some embodiments, the step of contacting the PSMA-expressing cancer cells with the mTOR inhibitor and the step of contacting the PSMA-expressing cancer cells with the PSMA ligand-anticancer agent conjugate are concurrent.

In some embodiments, the step of contacting the PSMA-expressing cancer cells with the mTOR inhibitor and the step of contacting the PSMA-expressing cancer cells with the PSMA ligand-anticancer agent conjugate are sequential.

In some embodiments, the step of contacting the PSMA-expressing cancer cells with the mTOR inhibitor is prior to the step of contacting the PSMA-expressing cancer cells with the PSMA ligand-anticancer agent conjugate.

In some embodiments, the PSMA-expressing cancer cells are in contact with the mTOR inhibitor for at least 7 days.

In some embodiments, the PSMA-expressing cancer cells comprise androgen-independent PSMA-expressing cancer cells.

In some embodiments, the PSMA-expressing cancer cells comprise PSMA-expressing cancer cells insensitive to antiandrogen therapy.

In some embodiments, the PSMA-expressing cancer cells insensitive to antiandrogen therapy are re-sensitized to antiandrogen therapy after the step of contacting the PSMA-expressing cancer cells with the mTOR inhibitor.

In some embodiments, the PSMA-expressing cancer cells may be of a tumor.

In some embodiments, the PSMA-expressing cancer cells are PSMA-expressing prostate cancer cells. In other embodiments, the PSMA-expressing cancer cells are PSMA-expressing non-prostate cancer cells. In yet other embodiments, the PSMA-expressing cancer cells are of the neovasculature of a non-prostate cancer or non-prostate tumor.

In some embodiments, the PSMA-expressing cancer cells are of a subject. In some embodiments, the subject has progressive metastatic castration-resistant prostate cancer. In some embodiments, the subject has had prior chemotherapy with one or more taxanes. In some embodiments, the subject has had prior treatment with one or more antiandrogens. In some embodiments, the subject has prostate cancer that has progressed despite prior treatment, such as with one or more antiandrogens. In some embodiments, the subject has prostate cancer that is starting to progress despite treatment, such as with one or more antiandrogens. In some embodiments, the subject has not had prior cytotoxic chemotherapy. In some embodiments, the subject has not had prior treatment with one or more antiandrogens. In some embodiments, the subject has not had prior treatment with enzalutamide or abiraterone. In some embodiments, the subject that has not had prior treatment with antiandrogens, such as enzalutamide or abiraterone, is one with metastatic castration-resistant prostate cancer. In some embodiments, the subject is any one of the subjects described herein.

In still other aspects of the disclosure provide methods of inhibiting proliferation of prostate-specific membrane antigen (PSMA)-expressing cancer cells. The methods may comprise contacting PSMA-expressing cancer cells with prednisone, and contacting the PSMA-expressing cancer cells with a PSMA ligand conjugate. In some embodiments, the method further comprises contacting the PSMA-expressing cancer cells with a compound that increases cell surface expression of PSMA, such as an antiandrogen.

In some embodiments, the antiandrogen blocks enzyme cytochrome CYP17. In some embodiments, the antiandrogen blocks enzyme cytochrome CYP17A1.

In some embodiments, the antiandrogen is an androgen receptor antagonist.

In some embodiments, the antiandrogen is abiraterone, enzalutamide, nilutamide, flutamide, bicalutamide, ARN 509, galeterone or orteronel. In some embodiments, the antiandrogen is enzalutamide or abiraterone.

In some embodiments, the antiandrogen is in an amount effective to upregulate PSMA expression.

In some embodiments, the step of contacting the PSMA-expressing cancer cells with prednisone and/or the compound that increases cell surface expression of PSMA, such as an antiandrogen, and the step of contacting the PSMA-expressing cancer cells with the PSMA ligand conjugate are concurrent. In other embodiments, the step of contacting the PSMA-expressing cancer cells with prednisone and/or the compound, such as an antiandrogen, and the step of contacting the PSMA-expressing cancer cells with the PSMA ligand conjugate are sequential.

In some embodiments, the step of contacting the PSMA-expressing cancer cells with prednisone and/or the compound, such as an antiandrogen, is prior to the step of contacting the PSMA-expressing cancer cells with the PSMA ligand conjugate.

In some embodiments, the step of contacting the PSMA-expressing cancer cells with the PSMA ligand conjugate occurs within one week of the step of contacting the PSMA-expressing cancer cells with the compound, such as an antiandrogen.

In some embodiments, the PSMA-expressing cancer cells are in contact with the compound, such as an antiandrogen, for at least 3 days. In other embodiments, the PSMA-expressing cancer cells are in contact with the compound, such as an antiandrogen, for at least 7 days. In yet other embodiments, the PSMA-expressing cancer cells are in contact with the compound, such as an antiandrogen, for at least 14 days. In still other embodiments, the PSMA-expressing cancer cells are in contact with the compound, such as an antiandrogen, for at least 21 days. In still other embodiments, the PSMA-expressing cancer cells are in contact with the compound, such as an antiandrogen, for at least 28 days.

In some embodiments, the PSMA-expressing cancer cells comprise androgen-independent PSMA-expressing cancer cells.

In some embodiments, the PSMA-expressing cancer cells comprise PSMA-expressing cancer cells insensitive to antiandrogen therapy.

In some embodiments, the PSMA-expressing cancer cells insensitive to antiandrogen therapy are re-sensitized to antiandrogen therapy after the step of contacting the PSMA-expressing cancer cells with the compound, such as an antiandrogen.

In some embodiments, the PSMA-expressing cancer cells are of a tumor.

In some embodiments, the PSMA-expressing cancer cells are PSMA-expressing prostate cancer cells. In other embodiments, the PSMA-expressing cancer cells are PSMA-expressing non-prostate cancer cells. In yet other embodiments, the PSMA-expressing cancer cells are of the neovasculature of a non-prostate cancer or non-prostate tumor.

In some embodiments, the PSMA-expressing cancer cells are of a subject. In some embodiments, the subject has progressive metastatic castration-resistant prostate cancer. In some embodiments, the subject has had prior chemotherapy with one or more taxanes. In some embodiments, the subject has had prior treatment with one or more antiandrogens. In some embodiments, the subject has prostate cancer that has progressed despite prior treatment, such as with one or more antiandrogens. In some embodiments, the subject has prostate cancer that is starting to progress despite treatment, such as with one or more antiandrogens. In some embodiments, the subject has not had prior cytotoxic chemotherapy. In some embodiments, the subject has not had prior treatment with one or more antiandrogens. In some embodiments, the subject has not had prior treatment with enzalutamide or abiraterone. In some embodiments, the subject that has not had prior treatment with antiandrogens, such as enzalutamide or abiraterone, is one with metastatic castration-resistant prostate cancer. In some embodiments, the subject is any one of the subjects described herein.

Yet other aspects of the disclosure provide methods of specific delivery of a cytotoxic agent to PSMA-expressing cells in a subject. The methods may comprise administering to a subject a compound that increases cell surface expression of PSMA, and administering to the subject a PSMA ligand-cytotoxic agent conjugate.

In some embodiments, the compound that increases cell surface expression of PSMA is an antiandrogen.

In some embodiments, the antiandrogen blocks enzyme cytochrome CYP17. In some embodiments, the antiandrogen blocks enzyme cytochrome CYP17A1.

In some embodiments, the antiandrogen is an androgen receptor antagonist. In some embodiments, the antiandrogen is abiraterone, enzalutamide, nilutamide, flutamide, bicalutamide, ARN 509, galeterone or orteronel. In some embodiments, the antiandrogen is enzalutamide or abiraterone.

In some embodiments, the compound that increases cell surface expression of PSMA is an mTOR inhibitor. In some embodiments, the mTOR inhibitor is rapamycin.

In some embodiments, the step of administering the compound and the step of administering the PSMA ligand-cytotoxic agent conjugate are concurrent. In other embodiments, the step of administering the compound and the step of administering the PSMA ligand-cytotoxic agent conjugate are sequential. In some embodiments, the step of administering the compound is prior to the step of administering the PSMA ligand-cytotoxic agent conjugate.

In some embodiments, the step of administering the PSMA ligand-cytotoxic agent conjugate occurs within one week of the step of administering the compound.

In some embodiments, the PSMA-expressing cells are in contact with the compound for at least 3 days. In other embodiments, the PSMA-expressing cells are in contact with the compound for at least 7 days. In yet other embodiments, the PSMA-expressing cells are in contact with the compound for at least 14 days. In still other embodiments, the PSMA-expressing cells are in contact with the compound for at least 21 days. In yet other embodiments, the PSMA-expressing cells are in contact with the compound for at least 28 days.

In some embodiments, the PSMA-expressing cells comprise androgen-independent PSMA-expressing cancer cells.

In some embodiments, the PSMA-expressing cells comprise PSMA-expressing cancer cells insensitive to antiandrogen therapy.

In some embodiments, the PSMA-expressing cells insensitive to antiandrogen therapy are re-sensitized to antiandrogen therapy after the step of administering the compound.

In some embodiments, the PSMA-expressing cells are of a tumor.

In some embodiments, the PSMA-expressing cells are PSMA-expressing prostate cancer cells. In other embodiments, the PSMA-expressing cells are PSMA-expressing non-prostate cancer cells. In still other embodiments, the PSMA-expressing cells are of the neovasculature of a non-prostate cancer or non-prostate tumor.

In some embodiments, the PSMA-expressing cancer cells are of a subject. In some embodiments, the subject has progressive metastatic castration-resistant prostate cancer. In some embodiments, the subject has had prior chemotherapy with one or more taxanes. In some embodiments, the subject has had prior treatment with one or more antiandrogens. In some embodiments, the subject has prostate cancer that has progressed despite prior treatment, such as with one or more antiandrogens. In some embodiments, the subject has prostate cancer that is starting to progress despite treatment, such as with one or more antiandrogens. In some embodiments, the subject has not had prior cytotoxic chemotherapy. In some embodiments, the subject has not had prior treatment with one or more antiandrogens. In some embodiments, the subject has not had prior treatment with enzalutamide or abiraterone. In some embodiments, the subject that has not had prior treatment with antiandrogens, such as enzalutamide or abiraterone, is one with metastatic castration-resistant prostate cancer. In some embodiments, the subject is any one of the subjects described herein.

Still other aspects of the disclosure provide compounds that increase cell surface expression of PSMA and a PSMA ligand-anticancer agent conjugate or PSMA ligand-cytotoxic agent conjugate. In yet other aspects, compositions are provided the comprise prednisone and a PSMA ligand conjugates as provided herein. In some embodiments, the compositions further comprise a compound that increases cell surface expression of PSMA.

In some embodiments, the compound that increases cell surface expression of PSMA is an antiandrogen.

In some embodiments, the antiandrogen blocks enzyme cytochrome CYP17. In some embodiments, the antiandrogen blocks enzyme cytochrome CYP17A1.

In some embodiments, the antiandrogen is an androgen receptor antagonist. In some embodiments, the antiandrogen is abiraterone, enzalutamide, nilutamide, flutamide, bicalutamide, ARN 509, galeterone or orteronel. In some embodiments, the antiandrogen is enzalutamide or abiraterone.

In some embodiments, the compound that increases cell surface expression of PSMA is an mTOR inhibitor. In some embodiments, the mTOR inhibitor is rapamycin.

In some embodiments, the compositions further comprise a pharmaceutically acceptable carrier and/or excipient.

Still other aspects of the disclosure provide kits that may comprise a container containing a compound that increases cell surface expression of PSMA in a PSMA-expressing cell, and a container containing a PSMA ligand-anticancer agent conjugate or PSMA ligand-cytotoxic agent conjugate. In other aspects, kits are provided that comprise a container containing prednisone, and a container containing a PSMA ligand conjugate. In some embodiments, the kit further comprises a container that comprises a compound that increases cell surface expression of PSMA.

In some embodiments, the compound that increases cell surface expression of PSMA is an antiandrogen.

In some embodiments, the antiandrogen blocks enzyme cytochrome CYP17. In some embodiments, the antiandrogen blocks enzyme cytochrome CYP17A1.

In some embodiments, the antiandrogen is an androgen receptor antagonist. In some embodiments, the antiandrogen is abiraterone, enzalutamide, nilutamide, flutamide, bicalutamide, ARN 509, galeterone or orteronel. In some embodiments, the antiandrogen is enzalutamide or abiraterone.

In some embodiments, the compound that increases cell surface expression of PSMA is an mTOR inhibitor. In some embodiments, the mTOR inhibitor is rapamycin.

In some embodiments, the kits further comprise a pharmaceutically acceptable carrier and/or excipient.

In some embodiments, the compound and/or the PSMA ligand conjugate is in an aqueous medium. In other embodiments, the compound and/or the PSMA ligand conjugate are/is lyophilized.

In some embodiments, the kits further comprise a diluent.

In some embodiments, the kits further comprise instructions for reconstituting the compound and/or the PSMA ligand conjugate. In other embodiments, the kits further comprise instructions for reconstituting prednisone and/or the PSMA ligand conjugate and/or the compound (when such kits further comprise the compound).

In some embodiments, the kits further comprise instructions for combining the compound and/or the PSMA ligand conjugate. In other embodiments, the kits further comprise instructions for combining prednisone and/or the PSMA ligand conjugate. In yet other embodiments, the kits further comprise instructions for combining the compound with prednisone and/or the PSMA ligand conjugate (when such kits further comprise the compound).

In some aspects, a method of inhibiting proliferation of prostate-specific membrane antigen (PSMA)-expressing cancer cells is provided. In some embodiments, the method comprises contacting PSMA-expressing cancer cells with prednisone; and contacting the PSMA-expressing cancer cells with a PSMA ligand conjugate. In embodiments of these methods, the PSMA-expressing cancer cells may be any one of the cells provided herein. In other embodiments, the PSMA ligand conjugate is any one of the PSMA ligand conjugates provided herein. In other aspects, a composition comprising prednisone and a PSMA ligand conjugate is provided. In still other aspects, a kit comprising a container containing prednisone, and a container containing a PSMA ligand conjugate is provided.

In some embodiments of any one of these methods, compositions or kits, the PSMA ligand of the conjugate comprises an antibody, or antigen-binding fragment thereof, that binds specifically PSMA. In such embodiments, the PSMA ligand is any one of the antibodies or antigen-binding fragments provided herein.

In some embodiments of any one of these methods, compositions or kits, the PSMA ligand of the conjugate comprises a small molecule ligand that binds specifically PSMA. In such embodiments, the PSMA ligand is any one of the small molecule ligands provided herein.

In some embodiments of any one of these methods, compositions or kits, the PSMA ligand conjugate comprises MIP-1095 or MIP-1072 conjugated to a cytotoxic radionuclide selected from the group consisting of I¹²³, I¹²⁵, I¹³¹, I¹²⁴ Br⁷⁵, Br⁷⁷ and F¹⁸.

In some embodiments of any one of these methods, compositions or kits, the anticancer or cytotoxic agent of the PSMA ligand conjugate is any one of the anticancer or cytotoxic agents provided herein. In some embodiments, the agent comprises an auristatin, tubulysin, a pyrrolobenzodiazepine dimer, calicheamicin, colchicine, ispinesib, combrestatin A4, maytansinoid DM1, maytansinoid DM4, doxorubicin, or a cytotoxic radionuclide. In some embodiments, the auristatin comprises monomethylauristatin norephedrine or monomethylauristatin phenylalanine.

In some embodiments of any one of these methods, the method further comprises contacting the PSMA expressing cells with a compound that increases cell surface expression of PSMA. In some embodiments of any one of these compositions or kits, the compositions or kits further comprise a compound that increases cell surface expression of PSMA. In some embodiments, the compound is an antiandrogen or an mTOR inhibitor. In such embodiments, the antiandrogen can be any one of the antiandrogens provided herein. In some embodiments, the mTOR inhibitor is any one of the mTOR inhibitors provided herein

In some embodiments of any one of these methods, compositions or kits, the antiandrogen is abiraterone, enzalutamide, nilutamide, flutamide, bicalutamide, ARN 509, galeterone or orteronel. In some embodiments, the antiandrogen is enzalutamide, abiraterone or ARN 509.

In some embodiments of any one of the methods provided, the steps of contacting are concurrent. In other embodiments, the steps of contacting are sequential. In some embodiments, the step of contacting the PSMA-expressing cancer cells with the prednisone and/or compound is prior to the step of contacting the PSMA-expressing cancer cells with the PSMA ligand conjugate.

In some embodiments of any one of these methods, compositions or kits, the PSMA-expressing cancer cells comprise androgen-independent PSMA-expressing cancer cells. In some embodiments, the PSMA-expressing cancer cells comprise PSMA-expressing cancer cells insensitive to antiandrogen therapy.

In some embodiments of any one of these methods, the PSMA-expressing cancer cells are of a subject. In such embodiments, the subject can be any one of the subjects provided herein.

In some embodiments of any one of the foregoing methods or compositions or kits, the antiandrogen is enzalutamide, abiraterone or ARN 509, and the PSMA ligand-anticancer agent conjugate is a PSMA ADC.

In some embodiments of any one of the foregoing compositions or kits, the compositions or kits further comprise a pharmaceutically acceptable carrier and/or excipient.

In some embodiments of any one of the foregoing kits, any one or all of the components of the kits are in an aqueous medium. In some embodiments of any one of the foregoing kits, any one or all of the components of the kits are lyophilized.

In some embodiments of any one of the foregoing kits, the kits further comprise instructions for reconstituting any one or all of the components of the kits. In some embodiments, of any one of the foregoing kits, the kits further comprise instructions for combining any one or all of the components of the kits.

In some embodiments of any one of the foregoing compositions or kits, the compositions or kits further comprise a diluent.

In any one of the foregoing aspects or embodiments, the PSMA ligand of the conjugate may comprise an antibody, or antigen-binding fragment thereof, that binds specifically PSMA. In an embodiment, the PSMA ligand is an antibody or antigen-binding fragment thereof that binds the extracellular portion of PSMA. In another embodiment, the PSMA ligand is an antibody or antigen-binding fragment thereof that binds a PSMA protein dimer. In still another embodiment, the PSMA ligand is an antibody or antigen-binding fragment thereof that binds preferentially a PSMA protein dimer not a PSMA protein monomer. In a further embodiment, the antibody or antigen-binding fragment thereof binds with at least a 2-fold, 3-fold, 4-fold, 5-fold, 10-fold or more greater affinity to a PSMA protein dimer as compared to a PSMA protein monomer. In yet another embodiment, the PSMA protein dimer and PSMA protein monomer are in a non-denatured form, such as a native conformation.

In any one of the foregoing aspects or embodiments, the antibody or antigen-binding fragment thereof may be an antibody selected from the group consisting of: PSMA 3.7, PSMA 3.8, PSMA 3.9, PSMA 3.11, PSMA 5.4, PSMA 7.1, PSMA 7.3, PSMA 10.3, PSMA 1.8.3, PSMAA3.1.3, PSMAA3.3.1, Abgenix 4.248.2, Abgenix 4.360.3, Abgenix 4.7.1, Abgenix 4.4.1, Abgenix 4.177.3, Abgenix 4.16.1, Abgenix 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153.1, Abgenix 4.232.3, Abgenix 4.292.3, Abgenix 4.304.1, Abgenix 4.78.1 and Abgenix 4.152.1, or an antigen-binding fragment thereof.

In any one of the foregoing aspects or embodiments, the antibody or antigen-binding fragment thereof may comprise (i) the three complementarity determining regions of a heavy chain variable region comprising an amino acid sequence set forth as SEQ ID NO:15 and (ii) the three complementarity determining regions of a light chain variable region comprising an amino acid sequence set forth as SEQ ID NO: 17.

In any one of the foregoing aspects or embodiments, the antibody or antigen-binding fragment thereof may comprise (i) the three complementarity determining regions of a heavy chain variable region comprising an amino acid sequence set forth as SEQ ID NO:19 and (ii) the three complementarity determining regions of a light chain variable region comprising an amino acid sequence set forth as SEQ ID NO: 21.

In any one of the foregoing aspects or embodiments, the antibody or antigen-binding fragment thereof may comprise (i) the three complementarity determining regions of a heavy chain variable region comprising an amino acid sequence set forth as SEQ ID NO:23 and (ii) the three complementarity determining regions of a light chain variable region comprising an amino acid sequence set forth as SEQ ID NO: 25.

In any one of the foregoing aspects or embodiments, the antibody or antigen-binding fragment thereof may comprise (i) the three complementarity determining regions of a heavy chain variable region comprising an amino acid sequence set forth as SEQ ID NO:27 and (ii) the three complementarity determining regions of a light chain variable region comprising an amino acid sequence set forth as SEQ ID NO: 29.

In any one of the foregoing aspects or embodiments, the antibody or antigen-binding fragment thereof may comprise (i) the three complementarity determining regions of a heavy chain variable region comprising an amino acid sequence set forth as SEQ ID NO: 31 and (ii) the three complementarity determining regions of a light chain variable region comprising an amino acid sequence set forth as SEQ ID NO: 33.

In any one of the foregoing aspects or embodiments, the antibody or antigen-binding fragment thereof may be antibody PSMA 10.3, AB-PG1-XG1-006, AB-PG1-XG1-026, AB-PG1-XG1-051, AB-PG1-XG1-069, AB-PG1-XG1-077, or an antigen-binding fragment thereof.

In any one of the foregoing aspects or embodiments, the antibody or antigen-binding fragment thereof may be antibody E99, J415, J533 or J591 or an antibody produced by a hybridoma having ATCC Accession Number HB-12101, HB-12109, HB-12127 or HB-12126, or an antigen-binding fragment thereof.

In any one of the foregoing aspects or embodiments, the antibody or antigen-binding fragment thereof may be an antibody produced by a hybidoma having ATCC Accession Number HB12060 (3F5.4G6), HB12309 (3D7-1.1), HB12310 (4E10-1.14), HB12489 (1G3), HB12495 (1G9), HB12490 (2C7), HB12494 (3C4), HB12491 (3C6), HB12484 (3C9), HB12486 (3E6), HB12488 (3E11), HB12485 (3G6), HB12493 (4D4), HB12487 (4D8), HB12492 (4C8B9), HB12664 (3F6), HB12678 (2E4), HB12665 (3C2), HB12672 (2D4), HB12660 (4C8G8), HB12675 (2C4), HB12663 (4C11), HB12661 (1D11), HB12667 (4E8), HB12674 (2G5), HB12620 (4E6), HB12677 (1F4), HB12666 (2E3), HB12662 (3D8), HB12668 (4F8), HB12673 (3D2), HB12676 (1G7), HB12669 (3D4), HB12679 (5G10) or HB12671 (5E9), or an antigen-binding fragment thereof.

In any one of the foregoing aspects or embodiments, the PSMA ligand of the conjugate may comprise a small molecule ligand that binds specifically PSMA. In an embodiment, the small molecule ligand binds or inhibits an enzymatic site of PSMA. In a particular embodiment, the small molecule ligand binds or inhibits the glutamate carboxypeptidase II (CCPII) site on PSMA.

In any one of the foregoing aspects or embodiments, the small molecule ligand may comprise MIP-1095, MIP-1072, GL2 or DUPA.

In any one of the foregoing aspects or embodiments, the PSMA ligand-anticancer agent conjugate may comprise EC1069 or EC1719.

In any one of the foregoing aspects or embodiments, the PSMA ligand-anticancer agent conjugate may comprise BIND-014.

In any one of the foregoing aspects or embodiments, the PSMA ligand conjugate comprises MIP-1095 or MIP-1072 conjugated to a cytotoxic radionuclide selected from the group consisting of I¹²³, I¹²⁵, I¹³¹, I¹²⁴ Br⁷⁵, Br⁷⁷ and F¹⁸.

In any one of the foregoing aspects or embodiments, the PSMA ligand conjugate may comprise ¹²³I-MIP-1095 or ¹²³I-MIP-1072.

In any one of the foregoing aspects or embodiments, the anticancer agent may comprise an auristatin, tubulysin, a pyrrolobenzodiazepine dimer, calicheamicin, colchicine, ispinesib, combrestatin A4, maytansinoid DM1, maytansinoid DM4, doxorubicin, or a cytotoxic radionuclide.

In any one of the foregoing aspects or embodiments, the auristatin may comprise monomethylauristatin norephedrine or monomethylauristatin phenylalanine.

In some embodiments of any one of the methods provided, the subject has received treatment with an antiandrogen and has experienced no or minimal sensitivity to the antiandrogen therapy. In some embodiments of any one of the methods provided, the subject has experienced a loss of sensitivity to an antiandrogen therapy. A subject that is sensitive to antiandrogen therapy is one that experiences an appreciable reduction in the subject's cancer, such as a decrease in the number of cancer cells, such as a decrease in the number of circulating cancer cells or tumor size, or symptoms associated with the cancer. Sensitivity to antiandrogen therapy may be measured by, for example, determining the level of proliferation of the subject's cancer cells after treatment and, in some embodiments, may be compared to the level of proliferation of the cancer cells prior to the treatment. Sensitivity to antiandrogen therapy may be determined during a course of a treatment with an antiandrogen, and when the level of reduction in the subject's cancer no longer appreciably changes or the subject experiences an undesired increase in the progression of the cancer, the subject may be considered to have experienced a loss of sensitivity to the antiandrogen therapy. In some embodiments, an increase in a subject's cancer refers to an increase in the number of cancer cells or tumor size or to an increase in the symptoms associate with the cancer. Sensitivity to antiandrogen therapy may be determined using routine methods by a clinician.

In embodiments of any one of the methods provided herein the subject may be one that has no or minimal sensitivity to an antiandrogen therapy. Additionally, in embodiments of any one of the methods provided herein the subject is one that has experienced a loss of sensitivity to antiandrogen therapy. Any one of the methods and compositions provided herein can be used to sensitize or resensitize such subjects to treatment with an antiandrogen when administered concurrently or sequentially with the PSMA ligand conjugates provided herein.

Any one of the methods provided herein can, in some embodiments, include the step of identifying a subject that has no or minimal sensitivity to antiandrogen therapy or that has experienced a loss of sensitivity to antiandrogen therapy. Any one of the methods provided herein, in some embodiments, can include the step of assessing the level of sensitivity to antiandrogen therapy in a subject. Any one of the methods provided herein, in some embodiments, can include the step of administering an antiandrogen until there is a loss of sensitivity thereto as well as a step of administering an antiandrogen concurrently or sequentially with a PSMA ligand conjugate as provided herein.

Any one of the methods provided herein can include a step of assessing the progression of the cancer in the subject. Progression can be assessed in a number of ways. In some embodiments, progression is assessed by any one or more of the following: determining the level of PSA, assessing bone metastases and assessing measurable disease. In some embodiments, progression of bone metastases is the appearance of 2 or more new bone lesions on a bone scan (CT scan, MRI), or new radiographic lesions. Progression may also be determined by RECIST (Eisenhauer E A et al Eur J Cancer 2009:45(2):228-47). In other embodiments, progression of the cancer is occurring when there is an increase in pain, such as bone pain. In any one of the methods provided herein, assessing progression can comprise assessing any one or more of the foregoing.

In some embodiments of any one of the methods provided herein, the PSMA-expressing cancer cells are of a subject. In some embodiments, the subject has progressive metastatic castration-resistant prostate cancer. In some embodiments, the subject has had prior chemotherapy with one or more taxanes. In some embodiments, the subject has had prior treatment with one or more antiandrogens. In some embodiments, the subject has prostate cancer that has progressed despite prior treatment, such as with one or more antiandrogens. In some embodiments, the subject has prostate cancer that is starting to progress despite treatment, such as with one or more antiandrogens. In some embodiments, the subject has not had prior cytotoxic chemotherapy. In some embodiments, the subject has not had prior treatment with one or more antiandrogens. In some embodiments, the subject has not had prior treatment with enzalutamide or abiraterone. In some embodiments, the subject that has not had prior treatment with antiandrogens, such as enzalutamide or abiraterone, is one with metastatic castration-resistant prostate cancer. In some embodiments, the subject is any one of the subjects described herein.

### BRIEF DESCRIPTION OF THE DRAWINGS

FIG. 1A presents a schematic showing the structure of two examples of antiandrogens that increase the expression of PSMA, enzalutamide and abiraterone; FIG. 1B presents a schematic showing the chemical structure of ARN-509 (MW=477.43); FIG. 1C presents a schematic showing the chemical structure of galeterone (TOK-001) (MW=388.55); FIG. 1D presents a schematic showing the structure of a PSMA ligand conjugate that includes a PSMA antibody conjugated to monomethyl auristatin E (MMAE) (PSMA antibody-drug conjugate (PSMA ADC)), an average of four molecules of MMAE in this embodiment.

FIG. 2 shows percent cell proliferation inhibition values and Bliss differences for LNCaP cells (A) and C4-2 cells (B) contacted with combinations of PSMA ligand conjugate and enzalutamide, abiraterone or rapamycin. Each data point represents the mean of three to seven independent assays. Bliss differences are depicted via heat maps of values that are positive (medium gray), negative (dark gray), or near zero (light gray). Bliss parameters that are significantly different from zero (P<0.05) are highlighted via bold text and dark gray borders, and the corresponding percent inhibition values are highlighted with shading and dark gray text. NA=not applicable. (C) Graph showing increase in PSMA expression and inhibition of cell proliferation as a function of increasing concentration of enzalutamide.

FIG. 3 shows percent cell proliferation inhibition values and Bliss differences for LNCaP cells (A) and C4-2 cells (B) contacted with combinations of microtubule inhibitors and antiandrogens. Each data point represents the mean of three to five independent assays. Bliss differences are depicted via heat maps of values that are positive (medium gray), negative (dark gray), or near zero (light gray). Bliss parameters that are significantly different from zero (P<0.05) are highlighted via bold text and dark gray borders, and the corresponding percent inhibition values are highlighted with shading and dark gray text. NA=not applicable.

FIG. 4 shows percent cell proliferation inhibition values and Bliss differences for LNCaP cells (A) and C4-2 cells (B) contacted with combinations of rapamycin with MMAE or enzalutamide. Each data point represents the mean of three independent assays. Bliss differences are depicted via heat maps of values that are positive (medium gray), negative (dark gray), or near zero (light gray). Bliss parameters that are significantly different from zero (P<0.05) are highlighted via bold text and dark gray borders, and the corresponding percent inhibition values are highlighted with shading and dark gray text. NA=not applicable.

FIG. 5 shows percent cell proliferation inhibition values and Bliss differences for LNCaP cells (A) and C4-2 cells (B) contacted with combinations of PSMA monoclonal antibody and enzalutamide, abiraterone or rapamycin. Each data point represents the mean of two or three independent assays. Bliss differences are depicted via heat maps of values that are positive (medium gray), negative (dark gray), or near zero (light gray). No Bliss parameters were significantly different from zero (P<0.05). NA=not applicable.

FIG. 6 shows a flow cytometry analysis of PSMA expression. PSMA expression was measured by flow cytometry on cells treated for 7 days with varying concentrations of enzalutamide, abiraterone or rapamycin (A-F) or treated with 1 μM enzalutamide for varying times (G). In A-D, anti-PSMA staining is shown as a function of antiandrogen concentrations as follows: green (0.125 μM), pink (0.5 μM), cyan (1 μM) and orange (5 μM). In E-F, the green, pink and cyan histograms represent rapamycin concentrations of 1, 10 and 100 nM, respectively. Filled purple histograms denote untreated cells, and the blue line depicts background staining with isotype-control antibody. (G) Time-course of PSMA expression in enzalutamide-treated C4-2 cells. Vertical bars depict mean fluorescence intensity (MFI) values on the left axis, and the red line represents the fold increase in PSMA expression in treated cells relative to untreated cells on the right axis. (PR=days post enzalutamide removal.)

FIG. 7 shows a Western blot protein expression analysis of whole-cell extracts of LNCaP (A) or C4-2 (B) cells left untreated (Unt) or treated for 7 days with enzalutamide (Enza), abiraterone (Abi) or rapamycin (Rapa) prior to the analysis.

FIG. 8 shows percent cell proliferation inhibition values and Bliss differences for LNCaP cells (A) and C4-2 cells (B) contacted with combinations of PSMA ligand conjugate and prednisone or inhibitors of Akt, PI3K or kinesin spindle protein (inhibitors MK-2206, GDC-0941 and SB743921, respectively). Each data point represents the mean of four or five independent assays. Bliss differences are depicted via heat maps of values that are positive (medium gray), negative (dark gray), or near zero (light gray). Bliss parameters that are significantly different from zero (P<0.05) are highlighted via bold text and dark gray borders, and the corresponding percent inhibition values are highlighted with shading and dark gray text. NA=not applicable.

FIG. 9 presents a schematic showing a chemical structure with a D-γ-Glu D-Asp-D-Phe-D-Cys linker.

FIG. 10 shows percent cell proliferation inhibition values and Bliss differences for LNCaP cells (A) and C4-2 cells (B) contacted with combinations of PSMAsmall molecule ligand conjugate, EC1069 and enzalutamide. Each data point represents one independent assay. Bliss differences are depicted via heat maps of values that are positive (medium gray), negative (dark gray), or near zero (light gray). NA=not applicable.

FIG. 11 presents a schematic showing the structure of PSMA small molecule ligand conjugate that comprises an anticancer agent, tubulysin (also referred to herein as EC1069).

FIG. 12 presents data from combinations of PSMA ADC with ARN-509 (Aragon Pharmaceuticals, androgen receptor inhibitor) and TOK-001 (Tokai Pharmaceuticals, CYP17 inhibitor and androgen receptor antagonist). Percent inhibition values and Bliss differences are shown for LNCaP cells (A) and C4-2 cells (B). Bliss differences are depicted via heat maps of values that are positive (medium gray), negative (dark gray), or near zero (light gray). NA=not applicable. ARN-509 and TOK-001 were purchased from Selleck Chemicals.

FIG. 13 demonstrates the effects of enzalutamide on proliferation and PSMA expression. These data show that the effects on proliferation and PSMA expression occur at similar concentrations in LNCaP cells (but are uncoupled in C4-2 cells).

FIG. 14 demonstrates the rapid, significant and dose-dependent increases in PSMA expression with enzalutamide. The effects were exhibited in a clinically meaningful dose range and were seen for androgen-dependent (LNCaP) and androgen-independent (C4-2) cells.

FIG. 15 also demonstrates the increased expression of PSMA with enzalutamide. PSMA expression was maximal after 3 to 4 weeks of exposure but returned to basal levels one week after removal of enzalutamide.

FIG. 16 shows the synergistic anticancer activity of enzalutamide and PSMA ADC in vitro. Results are the average of four repetitions for each condition. Values in bold indicate statistically significant synergy.

FIG. 17 shows that PSMA ADC synergizes with enzalutamide in vitro. The synergy was shown in both androgen-dependent and androgen-independent cells and was associated with the increased expression of PSMA. Surprisingly, the synergy was observed even when enzalutamide alone had minimal antitumor activity.

### DETAILED DESCRIPTION OF THE INVENTION

The invention relates, at least in part, to combination treatments for PSMA-expressing cancer, such as prostate cancer and, in particular, progressive, metastatic prostate cancer. The treatments can include administration of at least one agent that targets prostate specific membrane antigen (PSMA) in combination with an agent that targets androgen receptors (ARs). The invention is based, at least in part, on the surprising discovery that antiandrogens (e.g., enzalutamide or abiraterone) significantly and reversibly augment PSMA expression and potentiate the activity of PSMA ligand conjugates. In androgen-independent cells, the effects on PSMA expression and conjugate activity were synergistic and uncoupled from any anti-proliferative effect of the antiandrogens. Synergistic inhibition of tumor cell growth was associated with an upregulation of PSMA expression by antiandrogens, even in cells that were refractory to treatment with antiandrogens alone. Co-treatment, in effect, can re-sensitize the cells to antiandrogen therapy.

Both enzalutamide and abiraterone potently synergized with PSMA ligand conjugates over a range of concentrations. Synergy was observed on cells that were both responsive and unresponsive to treatment with antiandrogens alone. In androgen-dependent LNCaP cells, the pharmacologic effects of antiandrogens (e.g., antiproliferative effects, effects on gene expression, and synergy with PSMA ligand conjugates) were observed over a similar range of clinically relevant concentrations. In androgen-independent C4-2 cells, effects on gene expression and synergy were uncoupled from any appreciable antiproliferative activity of either enzalutamide or abiraterone. Thus, these antiandrogens remain pharmacologically active for C4-2 cells; however, the cells have adopted compensatory survival mechanisms that allow them to proliferate in the presence of ongoing androgen receptor (AR) blockade.

Little to no synergy was observed between the antiandrogens and the components of the conjugates (i.e., free MMAE and unmodified PSMA mAb). Free MMAE showed additive to weakly synergistic effects when combined with the antiandrogens. There was modest synergy between the antiandrogens and docetaxel. The unmodified mAb showed no antiproliferative activity either alone or in combination with antiandrogens. Therefore, the strong synergy observed with PSMA ligand conjugates is specific to the conjugates and is neither a pass-through effect of its components nor generalizable to microtubule inhibitors as a class.

PSMA expression was doubled after 7 days treatment with enzalutamide, and 4-fold higher after 21 days. The magnitude of PSMA upregulation by enzalutamide or abiraterone approached that induced by charcoal-stripped serum, which is depleted in a range of hormones, cytokines, and growth factors (40). The findings suggest near-maximal androgen suppression in our system. The time course of expression was monitored in cells treated with enzalutamide. PSMA expression increased with continued treatment over three weeks and then rapidly returned to baseline upon removal of enzalutamide. The findings have implications for combining and sequencing potent antiandrogens and PSMA-targeted therapies in the clinic.

In addition, PSMA ligand conjugates synergized with a PI3K/mTOR pathway inhibitor via a multimodal mechanism involving increased PSMA expression and disruption of microtubule function. In C4-2 cells, rapamycin exhibited minimal single-agent activity but potentiated the activity of PSMA ligand conjugates, suggesting that PI3K pathway activation is an adaptive response to ADC treatment in C4-2 cells. Thus, the invention is also based, in part, on the surprising discovery that, in some instances mTOR inhibitor, such as rapamycin, activity synergized with the activity of PSMA ligand conjugates.

As used herein, a “PSMA ligand conjugate” comprises a molecule that binds specifically PSMA, such as an extracellular domain of PSMA, and is conjugated to a therapeutic agent. The therapeutic agent may be an anticancer agent or a cytotoxic agent. A “PSMA ligand,” therefore, herein refers to a molecule that specifically binds PSMA, as described herein. When a PSMA ligand is conjugated to an anticancer agent, the PSMA ligand conjugate is also referred to herein as a “PSMA ligand-anticancer agent conjugate”. When a PSMA ligand is conjugated to a cytotoxic agent, the PSMA ligand conjugate is also referred to herein as a “PSMA ligand-cytotoxic agent conjugate.”

As used herein, “PSMA-expressing cells” refers to cells that express PSMA or that can express PSMA (e.g., human PSMA). PSMA is a 100 kD Type II membrane glycoprotein expressed in prostate tissues (Horoszewicz et al., 1987, Anticancer Res. 7:927-935; U.S. Pat. No. 5,162,504). PSMA was characterized as a type II transmembrane protein having sequence homology with the transferrin receptor (Israeli et al., 1994, Cancer Res. 54:1807-1811) and with NAALADase activity (Carter et al., 1996, Proc. Natl. Acad. Sci. U.S.A. 93:749-753). PSMA is expressed in increased amounts in prostate cancer (Horoszewicz et al., 1987, Anticancer Res. 7:927-935; Rochon et al., 1994, Prostate 25:219-223; Murphy et al., 1995, Prostate 26:164-168; and Murphy et al., 1995, Anticancer Res. 15:1473-1479). PSMA expression in cancerous prostate is approximately 10-fold greater than that in normal prostate. Expression in normal prostate is approximately 10-fold greater than that in the brain and is 50- to 100-fold greater than that of the liver or kidney. In most normal tissues, no expression of PSMA is observed.

PSMA expression increases with disease progression, becoming highest in metastatic, hormone-refractory disease. In addition, PSMA is also abundantly expressed on the neovasculature of a variety of non-prostate tumors, including bladder, breast, colon, pancreas, sarcoma, melanoma, renal, liver, lung (e.g., non-small cell lung carcinoma), and kidney tumors, but not on normal vasculature. “PSMA-expressing cells,” therefore, include PSMA-expressing cancer cells such as prostate cancer cells as well as PSMA-expressing cells (e.g., endothelial cells) of the neovasculature of a number of non-prostate cancers or non-prostate tumors.

As used herein, an “androgen-dependent PSMA-expressing cell” refers to a PSMA-expressing cell, such as a cancer cell, that is responsive to antiandrogens. A cell is herein considered to be responsive to an antiandrogen if proliferation of the cell can be substantially inhibited by the antiandrogen.

As used herein, an “androgen-independent PSMA-expressing cell” refers to a PSMA-expressing cell, such as a cancer cell, that is non-responsive to antiandrogens, also referred to herein as a “PSMA-expressing cell insensitive to antiandrogen therapy.” A cell is herein considered to be non-responsive to an antiandrogen if proliferation of the cell is not substantially inhibited by the antiandrogen.

In some embodiments, PSMA-expressing cells insensitive to antiandrogen therapy are sensitized to antiandrogen therapy after the step of contacting the PSMA-expressing cells with a compound that increases cell surface expression of PSMA (e.g., antiandrogen or mTOR inhibitor). Such cells can then be further contacted with a PSMA ligand conjugate as provided herein, and such contact can occur concurrently or sequentially. Thus, in some embodiments, androgen-independent PSMA-expressing cells (e.g., cancer cells) that are otherwise non-responsive to antiandrogens may become responsive, and, therefore, sensitized to antiandrogens when contacted with an antiandrogen, or an mTOR inhibitor, and can be further contacted with a PSMA ligand conjugate to effect cell proliferation inhibition or cell killing. Proliferation of such “sensitized” PSMA-expressing cancer cells may be substantially inhibited as a result. Sensitization of androgen-independent PSMA-expressing cells to antiandrogens may occur, in some embodiments, within 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days or more of being contacted with a compound that increases cell surface expression of PSMA. In some embodiments, sensitization of androgen-independent PSMA-expressing cells to antiandrogens may occur in less than 2 days.

In some embodiments, PSMA-expressing cells are contacted with a compound that increases cell surface expression of PSMA for a period of time sufficient to increase (e.g., upregulate) expression of PSMA on the surface of the cells. A time sufficient to upregulate cell surface expression of PSMA can be determined by myriad protein expression assays, which are known in the art, including for example enzyme-linked immunosorbent assays (ELISAs) and Western blotting. In some embodiments, a time sufficient to upregulate cell surface expression of PSMA may be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days or more. In some embodiments, a time sufficient to upregulate cell surface expression of PSMA may be less than 1 day.

Examples of PSMA ligands for use as provided herein include, without limitation, antibodies or antigen binding fragments thereof as well as small molecule ligands that bind specifically PSMA and may act as substrate mimicks of enzymatic sites on PSMA. Antibodies that bind specifically to PSMA may be referred to herein as “PSMA antibodies.” Likewise, small molecule ligands that bind specifically PSMA may be referred to herein as “PSMA small molecule ligands.”

As used herein, “specific binding” refers to molecule (e.g., antibody) binding to a predetermined target (e.g., antigen), in this case PSMA (e.g., human PSMA). In some embodiments, that sequence of PSMA is set forth as SEQ ID NO: 1. Typically, the molecule binds with an affinity that is at least two-fold greater than its affinity for binding to a non-specific target (e.g., BSA, casein), which is a target other than PSMA, an isoform or variant of PSMA, or a closely-related target.

An antibody or an antigen-binding fragment thereof of a PSMA ligand conjugate may be any antibody or antigen-binding fragment thereof that binds PSMA (e.g., binds specifically to an epitope of PSMA). Examples of PSMA antibodies for use as provided herein include, without limitation, those listed in Table 1. Antigen-binding fragments of these antibodies are also examples of antigen-binding fragments for use in the methods and compositions as provided herein.

In some embodiments, the antibody is produced by hybridomas referred to herein. These hybridomas were deposited pursuant to, and in satisfaction of, the requirements of the Budapest Treaty on the International Recognition of the Deposit of Microorganisms for the Purposes of Patent Procedure with the American Type Culture Collection (“ATCC”), having the address 10801 University Boulevard, Manassas, Va. 20110-2209, as an International Depository Authority and given the Patent Deposit Designations (Table 1):

TABLE 1

| | | Patent | |
|---|---|---|---|
| | | Deposit | |
| | | Desig- | Date of |
| Antibody | Hybridoma/Plasmid | nation | Deposit |
| PSMA 3.7 | PSMA 3.7 | PTA-3257 | Apr. 5, 2001 |
| PSMA 3.9 | PSMA 3.9 | PTA-3258 | Apr. 5, 2001 |
| PSMA 3.11 | PSMA 3.11 | PTA-3269 | Apr. 10, 2001 |
| PSMA 5.4 | PSMA 5.4 | PTA-3268 | Apr. 10, 2001 |
| PSMA 7.1 | PSMA 7.1 | PTA-3292 | Apr. 18, 2001 |
| PSMA 7.3 | PSMA 7.3 | PTA-3293 | Apr. 18, 2001 |
| PSMA 10.3 | PSMA 10.3 | PTA-3347 | May 1, 2001 |
| | PSMA 10.3 | PTA-4413 | May 29, 2002 |
| | HC in | | |
| | pcDNA (SEQ ID | | |
| | NO: 7) | | |
| | PSMA 10.3 | PTA-4414 | May 29, 2002 |
| | Kappa in | | |
| | pcDNA (SEQ ID | | |
| | NO: 13) | | |
| PSMA 1.8.3 | PSMA 1.8.3 | PTA-3906 | Dec. 5, 2001 |
| PSMA A3.1.3 | PSMA A3.1.3 | PTA-3904 | Dec. 5, 2001 |
| PSMA A3.3.1 | PSMA A3.3.1 | PTA-3905 | Dec. 5, 2001 |
| Abgenix 4.248.2 | Abgenix 4.248.2 | PTA-4427 | Jun. 4, 2002 |
| Abgenix 4.360.3 | Abgenix 4.360.3 | PTA-4428 | Jun. 4, 2002 |
| Abgenix 4.7.1 | Abgenix 4.7.1 | PTA-4429 | Jun. 4, 2002 |
| Abgenix 4.4.1 | Abgenix 4.4.1 | PTA-4556 | Jul. 18, 2002 |
| Abgenix 4.177.3 | Abgenix 4.177.3 | PTA-4557 | Jul. 18, 2002 |
| Abgenix 4.16.1 | Abgenix 4.16.1 | PTA-4357 | May 16, 2002 |
| Abgenix 4.22.3 | Abgenix 4.22.3 | PTA-4358 | May 16, 2002 |
| Abgenix 4.28.3 | Abgenix 4.28.3 | PTA-4359 | May 16, 2002 |
| Abgenix 4.40.2 | Abgenix 4.40.2 | PTA-4360 | May 16, 2002 |
| Abgenix 4.48.3 | Abgenix 4.48.3 | PTA-4361 | May 16, 2002 |
| Abgenix 4.49.1 | Abgenix 4.49.1 | PTA-4362 | May 16, 2002 |
| Abgenix 4.209.3 | Abgenix 4.209.3 | PTA-4365 | May 16, 2002 |
| Abgenix 4.219.3 | Abgenix 4.219.3 | PTA-4366 | May 16, 2002 |
| Abgenix 4.288.1 | Abgenix 4.288.1 | PTA-4367 | May 16, 2002 |
| Abgenix 4.333.1 | Abgenix 4.333.1 | PTA-4368 | May 16, 2002 |
| Abgenix 4.54.1 | Abgenix 4.54.1 | PTA-4363 | May 16, 2002 |
| Abgenix 4.153.1 | Abgenix 4.153.1 | PTA-4388 | May 23, 2002 |
| Abgenix 4.232.3 | Abgenix 4.232.3 | PTA-4389 | May 23, 2002 |
| Abgenix 4.292.3 | Abgenix 4.292.3 | PTA-4390 | May 23, 2002 |
| Abgenix 4.304.1 | Abgenix 4.304.1 | PTA-4391 | May 23, 2002 |
| AB-PG1-XG1-006 | AB-PG1-XG1-006 | PTA-4403 | May 29, 2002 |
| | Heavy Chain (SEQ ID | | |
| | NO: 2) | | |
| | AB-PG1-XG1-006 | PTA-4404 | |
| | Light Chain (SEQ ID | | |
| | NO: 8) | | |
| AB-PG1-XG1-026 | AB-PG1-XG1-026 | PTA-4405 | May 29, 2002 |
| | Heavy Chain (SEQ ID | | |
| | NO: 3) | | |
| | AB-PG1-XG1-026 | PTA-4406 | |
| | Light Chain (SEQ ID | | |
| | NO: 9) | | |
| AB-PG1-XG1-051 | AB-PG1-XG1-051 | PTA-4407 | May 29, 2002 |
| | Heavy Chain (SEQ ID | | |
| | NO: 4) | | |
| | AB-PG1-XG1-051 | PTA-4408 | |
| | Light Chain (SEQ ID | | |
| | NO: 10) | | |
| AB-PG1-XG1-069 | AB-PG1-XG1-069 | PTA-4409 | May 29, 2002 |
| | Heavy Chain (SEQ ID | | |
| | NO: 5) | | |
| | AB-PG1-XG1-069 | PTA-4410 | |
| | Light Chain (SEQ ID | | |
| | NO: 11) | | |
| AB-PG1-XG1-077 | AB-PG1-XG1-077 | PTA-4411 | May 29, 2002 |
| | Heavy Chain (SEQ ID | | |
| | NO: 6) | | |
| | AB-PG1-XG1-077 | PTA-4412 | |
| | Light Chain (SEQ ID | | |
| | NO: 12) | | |

An antibody or an antigen-binding fragment thereof of a PSMA ligand conjugate, in some embodiments, may comprise: (i) any one of the heavy chains provided herein and (ii) any one of the light chains provided herein. In some embodiments, a PSMA antibody as provided herein may include (i) any one of the heavy chain variable regions provided herein and (ii) any one of the light chain variable regions provided herein. In some embodiments, PSMA antibodies include (i) the three complementarity determining regions of any one of the heavy chain variable regions provided herein and (ii) the three complementarity determining regions of any one of the light chain variable regions provided herein.

Plasmids encoding exemplary heavy and light chains of antibodies were also deposited with the ATCC and are shown in Table 1 above.

Also provided are antigen-binding fragments of any one of the foregoing antibodies for use as the PSMA ligands of the PSMA ligand conjugates provided herein.

As used herein, the names of the deposited hybridomas or plasmids may be used interchangeably with the names of the antibodies. It would be clear to one of skill in the art when the name is intended to refer to the antibody or when it refers to the plasmids or hybridomas that encode or produce the antibodies, respectively. Additionally, antibody names may be an abbreviated form of the name shown in Table 1. For instance, antibody AB-PG1-XG1-006 may be referred to as “AB-PG1-XG1-006,” “PG1-XG1-006,” “XG1-006,” or “006.” In another example, the antibody PSMA 4.232.3 may be referred to as “PSMA 4.232.3,” “4.232.3,” “4.232.3,” or “4.232.” It is intended that all of the variations in the name of the antibody refer to the same antibody and not a different one.

As used herein, a “coding region” refers to a region of a nucleotide sequence that encodes a polypeptide sequence; the coding region can include a region coding for a portion of a protein that is later cleaved off, such as a signal peptide.

Those of skill in the art will appreciate that nucleic acids and polypeptides as provided herein include nucleotide sequences and amino acid sequences as provided herein. In some instances, the nucleotide sequences and amino acid sequences may include sequences that encode, or that are, signal peptides. Each of these sequences with, or without, the portion of the sequence that encodes or is a signal peptide is contemplated herein.

In some embodiments, a PSMA antibody is encoded by a nucleic acid molecule or amino acid molecule that is highly homologous to the nucleic acid molecules or amino acid molecules provided herein, respectively. For example, homologous nucleic acid molecule or amino acid molecule may comprise a nucleotide sequence or amino acid sequence that is at least about 90% identical to a nucleotide sequence or amino acid sequence provided herein. As another example, a nucleotide sequence or amino acid sequence is at least about 95% identical, at least about 97% identical, at least about 98% identical, or at least about 99% identical to a nucleotide sequence or amino acid sequence as provided herein. Homology can be calculated using various, publicly available software, which are well known to one of ordinary skill in the art. Exemplary tools include the BLAST system available from the website of the National Center for Biotechnology Information (NCBI) at the National Institutes of Health.

In some embodiments, PSMA antibodies include the antibodies provided in U.S. Pat. Nos. 6,107,090, 6,649,163 and 6,962,981. Such antibodies are incorporated herein by reference. PSMA antibodies, therefore, include E99, J415, J533, and J591 monoclonal antibodies; monoclonal antibodies produced by hybridomas having ATCC Accession Numbers HB-12101, HB-12109, HB-12127 and HB-12126; and monoclonal antibodies produced by hybridomas having ATCC Accession Numbers HB12060 (3F5.4G6), HB12309 (3D7-1.1), HB12310 (4E10-1.14), HB12489 (1G3), HB12495 (1G9), HB12490 (2C7), HB12494 (3C4), HB12491 (3C6), HB12484 (3C9), HB12486 (3E6), HB12488 (3E11), HB12485 (3G6), HB12493 (4D4), HB12487 (4D8), HB12492 (4C8B9), HB12664 (3F6), HB12678 (2E4), HB12665 (3C2), HB12672 (2D4), HB12660 (4C8G8), HB12675 (2C4), HB12663 (4C11), HB12661 (1D11), HB12667 (4E8), HB12674 (2G5), HB12620 (4E6), HB12677 (1F4), HB12666 (2E3), HB12662 (3D8), HB12668 (4F8), HB12673 (3D2), HB12676 (1G7), HB12669 (3D4), HB12679 (5G10), and HB12671 (5E9). Antigen-binding fragments of these antibodies are also contemplated for use as PSMA ligands of the PSMA ligand conjugates provided herein.

Also provided herein are nucleic acid molecules encoding PSMA antibodies and antigen-binding fragments thereof and vectors comprising the nucleic acid molecules as described herein. The vectors provided can be used to transform or transfect host cells for producing PSMA antibodies and antigen-binding fragments thereof with the specificity described herein.

As used herein, “antibody” refers to a glycoprotein comprising at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, C_{H}1, C_{H}2 and C_{H}3. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDRs), interspersed with regions that are more conserved, termed framework regions (FRs). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “antigen-binding fragment” of an antibody refers to one or more portions of an antibody that retain the ability to bind specifically to an antigen (e.g., PSMA). The antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody.

As used herein, “isolated antibody” refers to an antibody that is substantially free of other antibodies having different antigenic specificities (e.g., an isolated antibody that specifically binds to PSMA is substantially free of antibodies that specifically bind antigens other than PSMA). An isolated antibody that specifically binds to an epitope, isoform or variant of PSMA may, however, in some embodiments, have cross-reactivity to other related antigens, e.g., from other species (e.g., PSMA species homologs). Moreover, an isolated antibody may, in some embodiments, be substantially free of other cellular material and/or chemicals.

Isolated antibodies of the invention encompass various antibody isotypes, such as IgG1, IgG2, IgG3, IgG4, IgM, IgA1, IgA2, IgAsec, IgD, IgE. As used herein, “isotype” refers to the antibody class (e.g., IgM or IgG1) that is encoded by heavy chain constant region genes. Antibodies can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)₂ fragment, and a Fv fragment.

Antibodies, in some embodiments, may be polyclonal, monoclonal, or a mixture of polyclonal and monoclonal antibodies. Antibodies can be produced by a variety of techniques well known in the art. Procedures for raising polyclonal antibodies are well known. Monoclonal antibody production may be effected by techniques that are also well known in the art.

As used herein, “monoclonal antibody” refers to a preparation of antibody molecules of single molecular composition. A monoclonal antibody displays a single binding specificity and affinity for a particular epitope.

In some embodiments, recombinant antibodies are contemplated for use as provided herein. As used herein, a “recombinant antibody” refers to an antibody that is prepared, expressed, created or isolated by recombinant means, such as an antibody isolated from an animal (e.g., a mouse) that is transgenic for another species' immunoglobulin genes, an antibody expressed using a recombinant expression vector transfected into a host cell, an antibody isolated from a recombinant, combinatorial antibody library, or an antibody prepared, expressed, created or isolated by any other means that involves splicing of immunoglobulin gene sequences to other DNA sequences.

In yet other embodiments, an antibody may be a chimeric or humanized antibody. As used herein, a “chimeric antibody” refers to an antibody that combines the murine variable or hypervariable regions with the human constant region or constant and variable framework regions. As used herein, “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications as provided herein.

In still other embodiments, monoclonal antibodies provided here may be modified to be in the form of a bispecific antibody or a multispecific antibody. As used herein, a “bispecific antibody” includes any agent, e.g., a protein, peptide, or protein or peptide complex, that has two different binding specificities which bind to, or interact with (a) a cell surface antigen and (b) an Fc receptor on the surface of an effector cell. As used herein, a “multispecific antibody” includes any agent, e.g., a protein, peptide, or protein or peptide complex, that has more than two different binding specificities that bind to, or interact with, (a) a cell surface antigen, (b) an Fc receptor on the surface of an effector cell, and (c) at least one other component. Accordingly, antibodies provided herein, in some embodiments, may be bispecific, trispecific, tetraspecific, or other multispecific antibodies directed to cell surface antigens, such as PSMA, and to Fc receptors on effector cells. “Bispecific antibodies” also includes diabodies. Diabodies are bivalent, bispecific antibodies in which the V_{H} and V_{L} domains are expressed on a single polypeptide chain and use a linker that is too short to allow for pairing between the two domains on the same chain, thereby forcing the domains to pair with complementary domains of another chain and creating two antigen-binding sites (see, e.g., Holliger, P., et al. (1993) Proc. Natl. Acad. Sci. USA 90:6444-6448; Poijak, R. J., et al. (1994) Structure 2:1121-1123).

A bispecific antibody can be formed of an antigen-binding region specific for the extracellular domain of PSMA and an antigen-binding region specific for an effector cell that has tumoricidal or tumor inhibitory activity. The two antigen-binding regions of the bispecific antibody are either chemically linked or can be expressed by a cell genetically engineered to produce the bispecific antibody. (See generally, Fanger et al., 1995 Drug News & Perspec. 8(3):133-137). Suitable effector cells having tumoricidal activity include, without limitation, cytotoxic T-cells (primarily CD8⁺ cells), and natural killer cells.

In some embodiments, antibodies provided herein are human antibodies. As used herein, “human antibodies” include antibodies having variable and constant regions derived from human germline immunoglobulin sequences. Human antibodies of the invention may include amino acid residues not encoded by human germline immunoglobulin sequences (e.g., mutations introduced by random or site-specific mutagenesis in vitro or by somatic mutation in vivo). The term “human antibodies”, as used herein, is not intended to include antibodies in which CDR sequences derived from the germline of another mammalian species, such as a mouse have been grafted onto human framework sequences (otherwise referred to herein as “humanized antibodies”). Human antibodies directed against PSMA can be generated using transgenic mice carrying parts of the human immune system rather than the mouse system. Human PSMA antibodies provided herein specifically bind cell-surface PSMA and/or rsPSMA (recombinant soluble PSMA, i.e., with the sequence of the extracellular portion of PSMA) with sub-nanomolar affinity. Human PSMA antibodies provided herein may have binding affinities of about 1×10⁻⁹M or less, about 1×10⁻¹⁰M or less, or 1×10⁻¹¹M or less. In some embodiments, the binding affinity of human PSMA antibodies as provided herein is less than about 5×10⁻¹⁰ M.

Antibodies or antigen-binding fragments thereof can be selected, for example, based on the following criteria, which are not intended to be exclusive:
1) binding to live cells expressing PSMA;
2) high affinity of binding to PSMA;
3) binding to a unique epitope on PSMA (to eliminate the possibility that antibodies with complimentary activities when used in combination would compete for binding to the same epitope);
4) opsonization of cells expressing PSMA;
5) mediation of growth inhibition, phagocytosis and/or killing of cells expressing PSMA in the presence of effector cells;
6) modulation (inhibition or enhancement) of NAALADase, folate hydrolase, dipeptidyl peptidase IV and/or γ-glutamyl hydrolase activities;
7) growth inhibition, cell cycle arrest and/or cytotoxicity in the absence of effector cells;
8) internalization of PSMA;
9) binding to a conformational epitope on PSMA;
10) minimal cross-reactivity with cells or tissues that do not express PSMA; and
11) preferential binding to dimeric forms of PSMA rather than monomeric forms of PSMA.

PSMA antibodies and antigen-binding fragments thereof provided herein typically meet one or more, and in some instances, all of the foregoing criteria.

In some embodiments, an isolated antibody or antigen-binding fragment thereof may be selected for its ability to bind live cells expressing PSMA. In order to demonstrate binding of monoclonal antibodies to live cells expressing the PSMA, flow cytometry can be used. Binding of the antibody or antigen-binding fragment thereof to live cells expressing PSMA can inhibit the growth of the cells or mediate cytolysis of the cells. Cytolysis can be complement mediated or can be mediated by effector cells. In some embodiments, the cytolysis is carried out in a living organism, preferably a mammal, and the live cell is a tumor cell. Examples of tumors that can be targeted by PSMA antibodies (e.g., PSMA antibody conjugates) provided herein, include any tumor that expresses PSMA such as, e.g., prostate, bladder, pancreas, lung, colon, kidney, melanomas and sarcomas. A tumor that expresses PSMA includes tumors with neovasculature expressing PSMA.

In some embodiments, a PSMA antibody, or antigen-binding fragment thereof, binds to and is internalized with PSMA expressed on cells. Thus, a PSMA ligand conjugate comprising a PSMA antibody may be internalized with PSMA expressed on cells. The mechanism by which this internalization occurs is not critical to the practice of the present invention. For example, the antibody or antigen-binding fragment thereof can induce internalization of PSMA.

In some embodiments, a PSMA antibody, or antigen-binding fragment thereof, binds to a conformational epitope within the extracellular domain of the PSMA molecule. Antibodies that bind to native protein but not denatured protein are those antibodies that bind conformational epitopes, and are preferred antibodies in some embodiments.

In other embodiments, a PSMA antibody, or antigen-binding fragment thereof, binds to a dimer-specific epitope on PSMA. Generally, antibodies or antigen-binding fragments thereof that bind to a dimer-specific epitope preferentially bind the PSMA dimer rather than the PSMA monomer. Antibodies that bind to the PSMA dimer but not to the monomeric PSMA protein are preferred antibodies in some embodiments.

Other PSMA antibodies, or antigen-binding fragments thereof, provided herein include antibodies that bind specifically to an epitope on PSMA defined by a second antibody. To determine the epitope, one can use standard epitope mapping methods known in the art.

In some embodiments, the PSMA antibody of a PSMA ligand conjugate is a monoclonal antibody that binds prostate specific membrane antigen (PSMA) protein dimer, PSMA protein dimer being a homodimer of PSMA protein monomer having the sequence of SEQ ID NO: 1, or an antigen-binding fragment thereof, wherein the antibody, or the antigen-binding fragment, (i) binds live cells and (ii) binds with at least a two-fold greater affinity to PSMA protein dimer than to PSMA protein monomer, as described in U.S. Pat. No. 8,114,965. The antibodies and antigen-binding fragments provided in U.S. Pat. No. 8,114,965 are specifically incorporated by reference herein, any one of which antibodies or antigen-binding fragments thereof may be the PSMA antibody or antigen-binding fragment of a PSMA ligand conjugate as provide herein.

In some embodiments, PSMA antibodies are conjugated to radioactive molecules. An example of such a PSMA ligand conjugate, thus, includes ¹⁷⁷Lu-J591, which contains monoclonal PSMA antibody J591 conjugated through 1,4,7,10-tetraazacyclododecane-1,4,7,10-tetraacetic acid (DOTA) to ¹⁷⁷Lutetium (¹⁷⁷Lu).

The PSMA ligand of a PSMA ligand conjugate may be any small molecule ligand that binds specifically PSMA. Such small molecule ligands may bind to the enzymatic site of PSMA in its native conformation. Also, such small molecule ligands may possess any one or more of the characteristics described above for PSMA antibody ligands.

In some embodiments, the small molecule ligand is based on a glutamate-urea-lysine heterodimer (e.g., glutamate-urea-lysine analog), or a glutamate-urea-glutamate based dimer, that binds specifically to an enzymatic site on PSMA. In some embodiments, such small molecule ligands are conjugated to a radionuclide as the anticancer, or cytotoxic, agent (e.g., cytotoxic radionuclide or radiotherapeutic isotope). Examples of PSMA ligand conjugates, thus, include glutamate-urea-amino acid based small molecule ligands conjugated to a radionuclide through an intervening linker such as ¹²³I-MIP-1095 (also referred to as ¹²³I-MIP-1466) and ¹²³I-MIP-1072 (Molecular Insight Pharmaceuticals, Inc.). Other examples of PSMA small molecule ligands and PSMA ligand conjugates can be found in U.S. Pat. No. 8,465,725 and U.S. Pat. No. 8,487,129 and are incorporated herein by reference. In some embodiments, I¹²³ may be substituted with other radiohalogens including those selected from the group consisting of I¹²⁵, I¹³¹, I¹²⁴, BR⁷⁵, BR⁷⁷ and F¹⁸.

The chemical structure of ¹²³I-MIP-1095 (i.e., ¹²³I—(S)-2-(3-((S)-1-carboxy-5-(3-(4-iodophenyl)ureido)pentyl)ureido)pentanedioic acid) is:

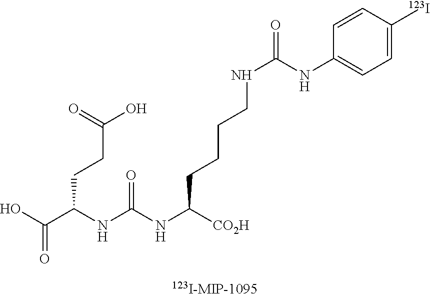 [SMILES: O=C(O)CC[C@H](NC(=O)N[C@@H](CCCCNC(=O)Nc1ccc([123I])cc1)C(=O)O)C(=O)O]

In another embodiment, the PSMA ligand conjugate is ¹²⁴I-MIP-1095. In another embodiment, the PSMA ligand conjugate is ¹³¹I-MIP-1095.

The chemical structure of ¹²³I-MIP-1072 (i.e., ¹²³I—(S)-2-(3-((S)-1-carboxy-5-(4-iodobenzylamino)pentyl)ureido)pentanedioic acid) is:

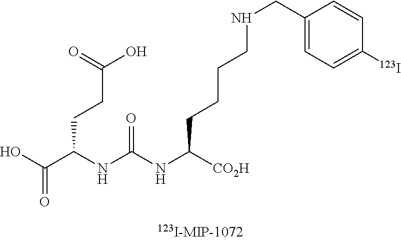 [SMILES: O=C(O)CC[C@H](NC(=O)N[C@@H](CCCCNCc1ccc([123I])cc1)C(=O)O)C(=O)O]

In some embodiments, the small molecule ligand of a PSMA ligand conjugate is a GL2 molecule as described in international Publication No. WO2010/005723. Any of the small molecules ligands provided herein, including a GL2 molecule, may be conjugated to a therapeutic agent by way of a nanoparticle (e.g., polymer-based, lipid-based and/or nucleic acid-based nanoparticles). In such embodiments, the nanoparticle may contain the therapeutic agent. Thus, in some embodiments, the PSMA ligand conjugate comprises a small molecule ligand conjugated to a nanoparticle that contains an anticancer or cytotoxic agent. Examples of such PSMA ligand conjugates include, without limitation, BIND-014 (Bind Biosciences, Inc.) described in International Publication No. WO2010/005723. The PSMA ligands and PSMA ligand conjugates of which are incorporated by reference herein.

PSMA small molecule ligands, in some embodiments, may be selected from the group consisting of compounds I, II, III and IV:

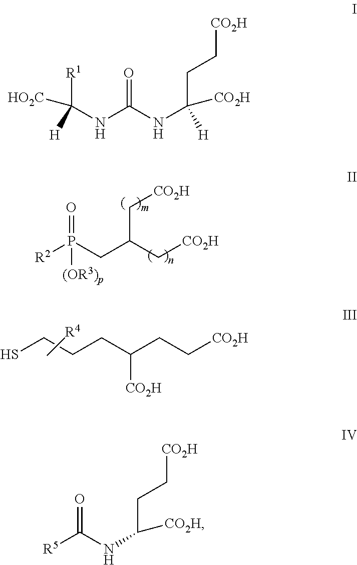

and enantiomers, stereoisomers, rotamers, tautomers, diastereomers, or racemates thereof; wherein m and n are each, independently, 0, 1, 2 or 3; p is 0 or 1; R1, R2, R4 and R5 are each, independently, selected from the group consisting of substituted or unsubstituted alkyl, substituted or unsubstituted aryl, and any combination thereof; and R3 is H or CH3; wherein R1, R2, R4 or R5 comprise a point of covalent attachment to the nanoparticle. For example, R1, R2, R4 and R5 may be each, independently, Ci₁₋₆-alkyl or phenyl, or any combination of Ci₁₋₆-alkyl or phenyl, which are independently substituted one or more times with OH, SH, NH2, or CO2H, and wherein the alkyl group may be interrupted by N(H), S or O. In some embodiments, for example, R1, R2, R4 and R5 are each, independently, CH₂-Ph, (CH₂)₂—SH, CH₂—SH, (CH₂)₂C(H)(NH₂)CO₂H, CH₂C(H)(NH₂)CO₂H, CH(NH₂)CH₂CO₂H, (CH₂)₂C(H)(SH)CO₂H, CH₂—N(H)-Ph, O—CH₂-Ph, or O—(CH₂)₂-Ph, wherein each Ph may be independently substituted one or more times with OH, NH₂, CO₂H or SH.

PSMA small molecule ligands, in other embodiments, may be selected from the group consisting of:

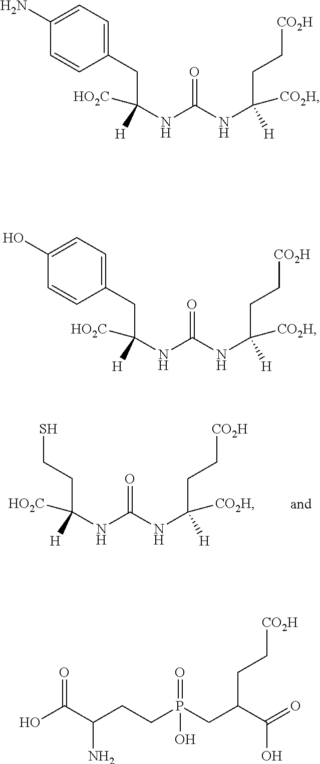 [SMILES: Nc1ccc(C[C@H](NC(=O)N[C@@H](CCC(=O)O)C(=O)O)C(=O)O)cc1 ; O=C(O)CC[C@H](NC(=O)N[C@@H](Cc1ccc(O)cc1)C(=O)O)C(=O)O ; O=C(O)CC[C@H](NC(=O)N[C@@H](CCS)C(=O)O)C(=O)O ; NC(CCP(=O)(O)CC(CCC(=O)O)C(=O)O)C(=O)O]

and enantiomers, stereoisomers, rotamers, tautomers, diastereomers, or racemates thereof; and wherein the NH₂, OH or SH groups serve as a point of covalent attachment, or may be selected from the group consisting of

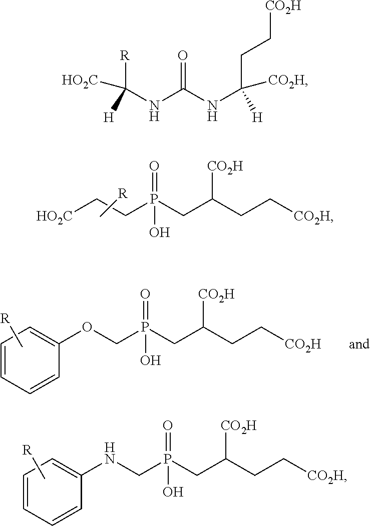

and enantiomers, stereoisomers, rotamers, tautomers, diastereomers, or racemates thereof; wherein R is independently selected from the group consisting of NH₂, SH, OH, CO₂H, Ci_{—}6-alkyl that is substituted with NH₂, SH, OH or CO₂H, and phenyl that is substituted with NH₂, SH, OH or CO₂H, and wherein R serves as the point of covalent attachment.

PSMA small molecule ligands, in yet other embodiments, may be selected from the group consisting of:

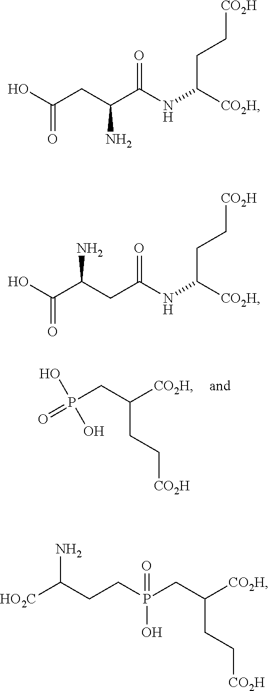

and enantiomers, stereoisomers, rotamers, tautomers, diastereomers, or racemates thereof; any of which may be further substituted with NH2, SH, OH, CO2H, Ci₁₋₆-alkyl that is substituted with NH₂, SH, OH or CO₂H, or phenyl that is substituted with NH₂, SH, OH or CO₂H, wherein these functional groups serve as the point of covalent attachment. For example, a low-molecular weight PSMA ligand may be

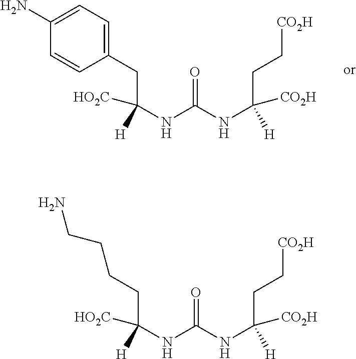 [SMILES: Nc1ccc(C[C@H](NC(=O)N[C@@H](CCC(=O)O)C(=O)O)C(=O)O)cc1 ; NCCCC[C@H](NC(=O)N[C@@H](CCC(=O)O)C(=O)O)C(=O)O]

and enantiomers, stereoisomers, rotamers, tautomers, diastereomers, or racemates thereof; wherein the NH₂ groups serve as the point of covalent attachment. Attachment may be to a linker, polymer, particle, etc.

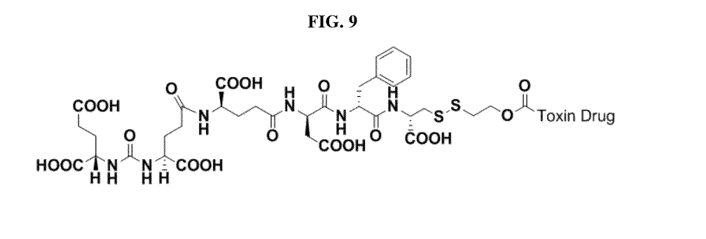

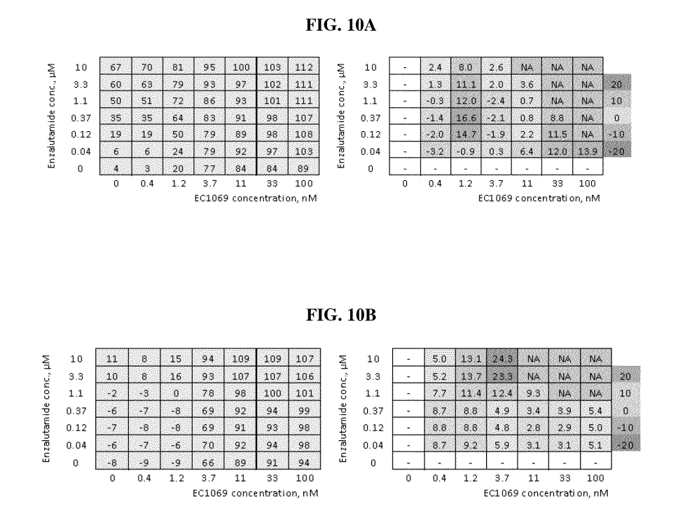

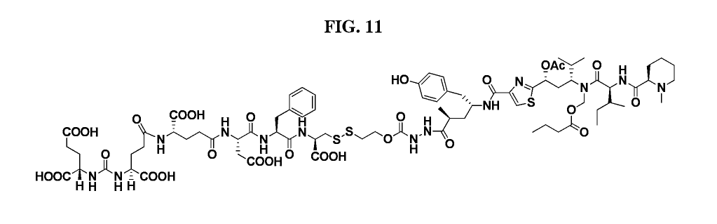

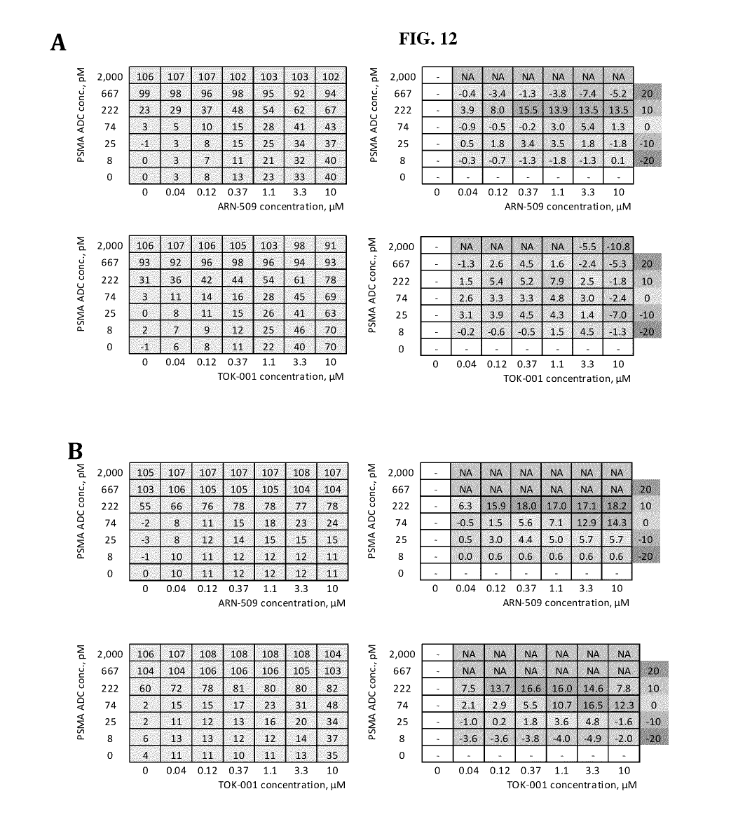

In some embodiments, the PSMA small molecule ligand that comprises a molecule that is or mimics a substrate that binds the enzymatic site on PSMA includes 2-[3-(1,3-dicarboxypropyl)ureido]pentanedioic acid (DUPA). In some embodiments, such small molecule ligands are conjugated to a chemotherapeutic agent, such as tubulysin hydrazide (TubH). The synthesis and uses of an example of such a PSMA ligand conjugate (EC1069) are described in Kularatne, S A et al J Med Chem 2010, 53, 7767-7777; Kularatne, S A et al Mol Phramaceutics Vol 6, no 3, 780-789, 2009. EC1719 is another example of a PSMA ligand conjugate that includes TubH. EC1069 and EC1719 can target the chemotherapy drug to PSMA receptors expressed on prostate cancer cells (Endocyte). Other EC1069 and EC1719 analogs linking DUPA and TubH can also target PSMA receptors expressed on prostate cancer cells. Thus, also contemplated herein are analogs of EC1069 and analogs of EC1719. The terms “EC1069” and “EC1719,” therefore, encompasses EC1069, EC1719 and analogs thereof. The linkers of the analogs, in some embodiments, may be peptides with D-amino-acid(s), or peptides attached with sugar moieties, amides or esters. An example of a linker, therefore, is D-γ-Glu D-Asp-D-Phe-D-Cys (FIG. 9). Other linkers may be used as provided herein.

Conjugation of one or more therapeutic agents to a PSMA ligand can include many chemical mechanisms, for instance covalent binding, affinity binding, intercalation, coordinate binding, electrostatic binding and complexation. Conjugation may also include encapsulation and is intended to refer to any mechanism by which one component may be associated with another component. Conjugation may be direct conjugation of the therapeutic agent to the PSMA ligand or it may be indirect, such as via a linker, polymer, particle etc., and it is the linker, polymer, particle, etc. to which the therapeutic agent is bound.

Covalent binding can be achieved either by direct condensation of existing side chains or by the incorporation of external bridging molecules. Many bivalent or polyvalent agents are useful in coupling protein molecules to other proteins, peptides or amine functions, etc. For example, the literature is replete with coupling agents such as carbodiimides, diisocyanates, glutaraldehyde, diazobenzenes, and hexamethylene diamines. This list is not intended to be exhaustive of the various coupling agents known in the art but, rather, is exemplary of the more common coupling agents.

In some embodiments, wherein the PSMA ligand is an antibody, it is contemplated the antibody is first derivatized, and then the therapeutic agent is attached to the derivatized product. Suitable cross-linking agents for use in this manner include, for example, SPDP (N-succinimidyl-3-(2-pyridyldithio)propionate), and SMPT, 4-succinimidyl-oxycarbonyl-methyl-(2-pyridyldithio)toluene.

In some embodiments, where the agent is a protein toxin, it may be fused to the PSMA ligand by genetic methods to form a hybrid immunotoxin fusion protein. The fusion proteins can include additional peptide sequences, such as peptide spacers that operatively attach, for example, the PSMA ligand and toxin, as long as such additional sequences do not appreciably affect the targeting or toxin activities of the fusion protein. The proteins, in some embodiments, may be attached by a peptide linker or spacer, such as a glycine-serine spacer peptide, or a peptide hinge, as is well known in the art. Thus, for example, if the PSMA ligand is a PSMA antibody, the C-terminus of PSMA antibody can be fused to the N-terminus of the protein toxin molecule to form an immunotoxin that retains the binding properties of the PSMA antibody. Other fusion arrangements will be known to one of ordinary skill in the art. To express the fusion immunotoxin, the nucleic acid encoding the fusion protein is inserted into an expression vector in accordance with standard methods, for stable expression of the fusion protein, such as in mammalian cells, such as CHO cells. The fusion protein can be isolated and purified from the cells or culture supernatant using standard methodology, such as a PSMA affinity column.

Examples of anticancer agents for use as provided herein include, without limitation, cytotoxic agents, chemotherapeutic agents and agents that act on tumor neovasculature. Cytotoxic agents include, but are not limited to, cytotoxic radionuclides, chemical toxins and protein toxins. Cytotoxic radionuclides or radiotherapeutic isotopes include alpha-emitting isotopes such as, for example, ²²⁵Ac, ²¹¹At, ²¹²Bi, ²¹³Bi, ²¹²Pb, ²²⁴Ra, ²²³Ra. Cytotoxic radionuclides or radiotherapeutic isotopes include beta-emitting isotopes such as, for example, ¹⁸⁶Rh, ¹⁸⁸Rh, ¹⁷⁷Lu, ⁹⁰Y, ¹³¹I, ⁶⁷Cu, ⁶⁴Cu, ¹⁵³Sm, ¹⁶⁶Ho. In some instances, cytotoxic radionuclides may emit Auger and/or low energy electrons and include the isotopes ¹²³I, ¹²⁴I, ¹²⁵I, ¹³¹I, ⁷⁵Br, ⁷⁷Br and ¹⁸F.

Radionuclides typically are coupled to an antibody or antigen-binding fragment thereof by chelation. For example, in the case of metallic radionuclides, a bifunctional chelator is commonly used to link the isotope to the antibody or other protein of interest. Typically, the chelator is first attached to the antibody, and the chelator-antibody conjugate is contacted with the metallic radioisotope. A number of bifunctional chelators have been developed for this purpose, including the diethylenetriamine pentaacetic acid (DTPA) series of amino acids described in U.S. Pat. Nos. 5,124,471, 5,286,850 and 5,434,287, which are incorporated herein by reference. As another example, hydroxamic acid-based bifunctional chelating agents are described in U.S. Pat. No. 5,756,825, the contents of which are incorporated herein. Another example is the chelating agent termed p-SCN-Bz-HEHA (1,4,7,10,13,16-hexaazacyclo-octadecane-N,N′,N″,N′″,N″″,N′″″-hexaacetic acid) (Deal et al., J. Med. Chem. 42:2988, 1999), which is an effective chelator of radiometals such as ²²⁵Ac. Yet another example is DOTA (1,4,7,10-tetraazacyclododecane N,N′,N″,N′″-tetraacetic acid), which is a bifunctional chelating agent (see McDevitt et al., Science 294:1537-1540, 2001) that can be used in a two-step method for labeling followed by conjugation.

Chemical toxins or chemotherapeutic agents include, but are not limited to, members of the enediyne family of molecules, such as calicheamicin and esperamicin. Chemical toxins or chemotherapeutic agents can also include pyrrolobenzodiazepine (PBD) dimers (e.g., SJG-136, SG2000, SG2202, SG2285 as described in Hartley J A et al., Cancer Res. 2010, 70(17):6849-58), calicheamicins, colchicine, ispinesib (a novel small molecule inhibitor of kinesin spindle protein), combrestatin (e.g., combrestatin A4), maytansine derivatives such as maytansinoid DM4 (N2′-deacetyl-N2′-(4-mercapto-4-methyl-1-oxopentyl)maytansine) and maytansinoid DM1 (mertansine), methotrexate, doxorubicin, melphalan, chlorambucil, ARA-C, vindesine, mitomycin C, cis-platinum, etoposide, bleomycin and/or 5-fluorouracil. Other antineoplastic agents include dolastatins (U.S. Pat. Nos. 6,034,065 and 6,239,104) and derivatives thereof. Dolastatins and derivatives thereof include dolastatin 10 (dolavaline-valine-dolaisoleuine-dolaproine-dolaphenine) and the derivatives auristatin PHE (dolavaline-valine-dolaisoleuine-dolaproine-phenylalanine-methyl ester) (Pettit, G. R. et al., Anticancer Drug Des. 13(4):243-277, 1998; Woyke, T. et al., Antimicrob. Agents Chemother. 45(12):3580-3584, 2001), aurastatin E (e.g., monomethylauristatin norephedrine), aurastatin F (e.g., monomethylauristatin phenylalanine) and the like. Toxins also include poisonous lectins, plant toxins such as ricin, abrin, modeccin, botulina and diphtheria toxins. Other chemotherapeutic agents are known to those skilled in the art and may be used as provided herein.

Agents that act on the tumor vasculature include, but are not limited to, tubulin-binding agents (e.g., anti-tubulin agents) such as tubulysin and derivatives thereof (Kaur et al., Biochem J. 396(Pt 2):235-242, 2006), combrestatin A4 (Griggs et al., Lancet Oncol. 2:82, 2001), angiostatin and endostatin (reviewed in Rosen, Oncologist 5:20, 2000, incorporated by reference herein) and interferon inducible protein 10 (U.S. Pat. No. 5,994,292). A number of other antiangiogenic agents are also contemplated and include: 2ME2, angiostatin, angiozyme, anti-VEGF RhuMAb, Apra (CT-2584), avicine, benefin, BMS275291, carboxyamidotriazole, CC4047, CC5013, CC7085, CDC801, CGP-41251 (PKC 412), CM101, combretastatin A-4 prodrug, EMD 121974, endostatin, flavopiridol, genistein (GCP), IM-862, ImmTher, interferon alpha, interleukin-12, gefitinib (ZD1839), marimastat, metastat (Col-3), neovastat, octreotide, paclitaxel, penicillamine, photofrin, photopoint, PI-88, prinomastat (AG-3340), PTK787 (ZK22584), RO317453, solimastat, squalamine, SU 101, SU 5416, SU-6668, suradista (FCE 26644), suramin (metaret), tetrathiomolybdate, thalidomide, TNP-470 and vitaxin. Additional antiangiogenic agents are described by Kerbel, J. Clin. Oncol. 19(18s):45s-51s, 2001, which is incorporated by reference herein. Such agents are contemplated for use in the PSMA ligand conjugates provided herein.

In some embodiments, a PSMA ligand conjugate is a PSMA antibody-drug conjugate. Non-limiting examples of PSMA antibody-drug conjugates are described in US-2007-0160617-A1 and US-2011-0250216-A, such examples of each of which are incorporated by reference herein. In some embodiments, a PSMA antibody-drug conjugate comprises an antibody or antigen-binding fragment thereof that specifically binds PSMA and is conjugated to a dolastatin 10 derivative, in particular auristatins such as, MMAE (also referred to herein as monomethylauristatin E or monomethylauristatin norephedrine) or MMAF (also referred to herein as monomethylauristatin F or monomethylauristatin phenylalanine).

The antibody or antigen-binding fragment thereof can be, in some embodiments, conjugated to MMAE or MMAF with a compound of the following formula (Formula 1): -Aₙ-Yₘ—Zₘ—Xₙ—Wₙ—, wherein A is a carboxylic acyl unit; Y is an amino acid; Z is an amino acid; X and W are each a self-immolative spacer; n is an integer of 0 or 1; and m is an integer of 0 or 1, 2, 3, 4, 5 or 6. The ADC, in some embodiments, is represented by the formula (Formula 2): L-{Aₙ-Yₘ—Zₘ—Xₙ—Wₙ-D}ₚ wherein L is an antibody or antigen-binding fragment thereof that binds PSMA, D is MMAE or MMAF and p is an integer of 1, 2, 3, 4, 5, 6, 7 or 8. The other components are as described above. In one embodiment, the carboxylic unit “Aₙ” is linked to the antibody or antigen-binding fragment via a sulfur atom derived from the antibody or antigen-binding fragment:

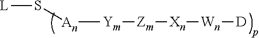

In one embodiment, A is

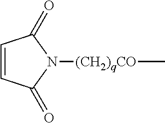

in which q is 1-10. Therefore, in one embodiment, the conjugate is:

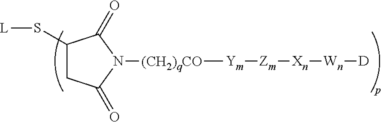

wherein L, Y, Z, X, W, D, n, m, q and p are as previously defined.

In another embodiment, A is 4-(N-succinimidomethyl)cyclohexane-1-carbonyl, m-succinimidobenzoyl, 4-(p-succinimidophenyl)-butyryl, 4-(2-acetamido)benzoyl, 3-thiopropionyl, 4-(1-thioethyl)-benzoyl, 6-(3-thiopropionylamido)-hexanoyl or maleimide caproyl. In a further embodiment, A is maleimide caproyl. Representative examples of various carboxylic acyl units and methods for their synthesis and attachment are described in U.S. Pat. No. 6,214,345, the entire contents of which, but in particular the examples and methods for their synthesis and attachment, are herein incorporated by reference.

In another embodiment, Y is alanine, valine, leucine, isoleucine, methionine, phenylalanine, tryptophan or proline. In yet another embodiment, Y is valine. In a further embodiment, Z is lysine, lysine protected with acetyl or formyl, arginine, arginine protected with tosyl or nitro groups, histidine, ornithine, ornithine protected with acetyl or formyl, or citrulline. In still a further embodiment, Z is citrulline. In one embodiment Yₘ—Zₘ is valine-citrulline. In another embodiment, Yₘ—Zₘ is a protein sequence which is selectively cleavable by a protease.

In a further embodiment, X is a compound having the formula

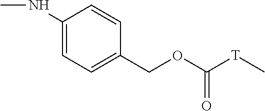

in which T is O, N, or S. In another embodiment, X is a compound having the formula —HN—R¹—COT in which R¹ is C₁-C₅ alkyl, T is O, N or S. In a further embodiment, X is a compound having the formula

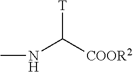

in which T is O, N, or S, R² is H or C₁-C₅ alkyl. In one embodiment, X is p-aminobenzylcarbamoyloxy. In another embodiment, X is p-aminobenzylalcohol. In a further embodiment, X is p-aminobenzylcarbamate. In yet a further embodiment, X is p-aminobenzyloxycarbonyl. In another embodiment, X is γ-aminobutyric acid; α,α-dimethyl γ-aminobutyric acid or β,β-dimethyl γ-aminobutyric acid.

In some embodiments, W is

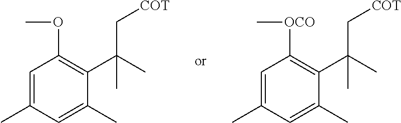

in which T is O, S or N.

In one embodiment, the compound of Formula 1 is maleimidocaproyl. Maleimidocaproyl has been used for conjugation of two specific auristatins to an anti-CD30 mAb (AC10) (Doronina, Svetlana et al. “Novel Linkers for Monoclonal Antibody-Mediated Delivery of Anticancer Agents”, AACR, Anaheim, Calif., Abstract No. 1421, Apr. 16-20, 2005). Maleimidocaproyl reacts with thiol groups to form a thioether.

MMAE or MMAF can be conjugated to an antibody or antigen-binding fragment thereof using methods known to those of ordinary skill in the art (e.g., See, Niemeyer, C M, Bioconjugation Protocols, Strategies and Methods, Humana Press, 2004) or as described herein. In some embodiments, more than one MMAE or MMAF molecule is conjugated to the antibody or antigen-binding fragment thereof. In other embodiments, 1, 2, 3, 4, 5, 6, 7 or 8 MMAE or MMAF molecules are conjugated to the antibody or antigen-binding fragment thereof. In still other embodiments, at least 2, 3, 4 or 5 MMAE or MMAF molecules are conjugated to the antibody or antigen-binding fragment thereof. In further embodiments, 2, 3, 4 or 5 MMAE or MMAF molecules are conjugated to the antibody or antigen-binding fragment thereof.

In some embodiments, the PSMA ligand conjugate is PSMA antibody (or antigen-binding fragment thereof)-maleimide caproyl-valine-citrulline-p-aminobenzyloxycarbonyl-monomethylauristatin norephedrine, PSMA antibody (or antigen-binding fragment thereof)-maleimide caproyl-valine-citrulline-p-aminobenzylcarbamate-monomethylauristatin norephedrine, PSMA antibody (or antigen-binding fragment thereof) -maleimide caproyl-monomethylauristatin norephedrine, PSMA antibody (or antigen-binding fragment thereof) -maleimide caproyl-valine-citrulline-p-aminobenzyloxycarbonyl-monomethylauristatin phenylalanine, PSMA antibody (or antigen-binding fragment thereof) -maleimide caproyl-valine-citrulline-p-aminobenzylcarbamate-monomethylauristatin phenylalanine or PSMA antibody (or antigen-binding fragment thereof) -maleimide caproyl-monomethylauristatin phenylalanine. In any of the foregoing, the PSMA antibody or antigen-binding fragment thereof may be any of the antibodies or antigen-binding fragments provided herein.

PSMA antibody-drug conjugates, for example, have been found to have particularly high levels of selectivity when killing of non-PSMA-expressing cells is compared to killing of PSMA-expressing cells. Therefore, in some embodiments, PSMA antibody-MMAE or PSMA antibody-MMAF conjugates have a PC-3™ cell to C4-2 cell or LNCaP™ cell selectivity of at least 250. In other embodiments, the selectivity is at least 300, 350, 400, 450, 500, 600, 700, 800, 900, 1000, 1100, 1200, 1300, 1400, 1500, 1600, 1700, 1800, 1900, 2000, 2250, 2500, 2750, 3000, 3500, 4000, 4500, 5000, 5500, 6000, 6500, 7000, 7500, 8000, 8500, 9000, 9500, 10000, 11000, 12000, 13000, 14000, 15000, 17500, 20000 or more. In some embodiments, the selectivity is between 250-500, 500-750, 750-1000, 1000-2000, 2000-5000, 5000-10000, 10000-15000 or 15000-20000. “Selectivity”, as defined herein, refers to the ratio of IC₅₀ values of a PSMA antibody-MMAE conjugate or PSMA antibody-MMAF conjugate on PC-3™ cells (non-PSMA-expressing cells) to C4-2 cells or LNCaP™ cells (PSMA-expressing cells).

PSMA antibody-drug conjugates, in some embodiments, mediate PSMA-expressing cell specific killing at very low concentrations, such as at or near picomolar concentrations. PSMA antibody-drug conjugates, in some embodiments, exhibit IC₅₀s at concentrations of less than about 1×10⁻¹⁰M, less than about 1×10⁻¹¹M, or less than about 1×10⁻¹²M. In some embodiments, an IC₅₀ is achieved at a concentration of less than about 1.5×10⁻¹¹M. In other embodiments, PSMA antibody-drug conjugates exhibit IC₅₀s of between 10-210, 40-210, 60-210 or 65-210 picomoles (pM). In yet other embodiments, PSMA antibody-drug conjugates exhibit IC₅₀s of about 10, 40, 60 or 80 pM. In still other embodiments, PSMA antibody-drug conjugates exhibit IC₅₀s of about 11, 42, 60 or 83 pM.

The level of cell killing can be determined by any of the methods provided herein or otherwise known to those of ordinary skill in the art.

An “antiandrogen,” as used herein, refers to an agent that blocks (e.g., inhibits) the action of androgen hormones and androgen-regulated molecules. Adrenergic receptor antagonists are herein considered to be antiandrogens. The term “antiandrogen” includes antiandrogens, antiandrogen analogs, and antiandrogen derivatives. In prostate cancer, antiandrogens block the activity of testosterone, which typically slows prostate cancer growth. In some embodiments, an antiandrogen blocks enzyme cytochrome P450 17A1, encoded by the CYP17A gene. Antiandrogens may be steroidal or non-steroidal (also referred to as “pure”). Examples of antiandrogens for use as provided herein include, without limitation, abiraterone (ZYTIGA®), enzalutamide (XTANDI®), nilutamide (NILANDRON®), flutamide (EULEXIN®), bicalutamide (CASODEX®), orteronel (TAK-700, Tokai Pharmaceuticals, Inc.) Potent antiandrogens such as, for example, enzalutamide, abiraterone, ARN 509 (Aragon Pharmaceuticals, Inc.) and galeterone (TOK-001 or VN/124-1, Tokai Pharmaceuticals, Inc.), which are typically used in progressive, metatstatic castration-resistant prostate cancer and which affect expression of a host of androgen-regulated molecules, such as PSMA expression.

“Potent” antiandrogens, or “second-generation” antiandrogens herein include antiandrogens that retain activity in a cellular environment of increased androgen receptor expression relative to wild-type expression. Potent antiandrogens include those that are active in subjects with castration-resistant prostate cancer. Potent antiandrogens also include antiandrogens that bind to the androgen receptor with greater relative affinity than common, clinically used antiandrgogens (e.g., bicalutamide, nilutamide, flutamide). Examples of potent antiandrogens include, without limitation, enzalutamide, abiraterone, ARN-509, galeterone, and diarylthiohydantoins RD162 and MDV310, 3beta-hydroxyandrosta-5,16-diene (HAD) and androsta-1,4-diene-3,17-dione-17-ethylene ketal (OAK) (Miyamoto H et al., Int J Cancer 2005, 117(5):866-72, incorporated by reference herein). Abiraterone, for example, inhibits the CYP17A1 enzyme. Enzalutamide, as another example, is an androgen receptor antagonist. As yet another example, galeterone is a selective CYP17 inhibitor and an androgen receptor antagonist. Methods that may be used to identify potent antiandrogens are described, for example, by Tran C et al., Science, 2009 324(5928):787-90, incorporated by reference herein. Any of the antiandrogens described herein may be used in any of the methods provided herein.

The compound that increases the cell surface expression of PSMA can also be an mTOR inhibitor. Examples of mTOR inhibitors for use as provided herein include, without limitation, rapamycin, everolimus (AFINITOR®, ZORTRESS®) and temsirolimus (TORISEL®). The term “mTOR inhibitor” includes mTOR inhibitors, mTOR inhibitor analogs, and mTOR inhibitor derivatives.

Also provided herein are compositions, for example, pharmaceutical compositions, which comprise a compound that increases cell surface expression of PSMA, a PSMA ligand conjugate, or a combination of the two. In some embodiments, a composition is administered to a subject (e.g., a mammal, such as a human). In some embodiments, a composition comprising a PSMA ligand conjugate is administered to a subject and another composition comprising a compound that increases cell surface expression of PSMA is separately administered to the subject, either sequentially or concurrently. Alternatively, in some embodiments, a composition comprising both a PSMA ligand conjugate and a compound that increases cell surface expression of PSMA is administered to a subject.

In some embodiments, a composition comprising a compound that increases cell surface expression of PSMA is first administered to a subject, and then a composition comprising a PSMA ligand conjugate is administered to the subject immediately after, within an hour, within a couple of hours, within a day, within 2 days, within 3 days, within 4 days, with 5 days, within 6 days, or within a week or more. Either composition may be administered once or more than once (e.g., twice, thrice, etc.)

As used herein, “a compound that increases cell surface expression of PSMA,” refers to a compound that increases the cell surface expression of PSMA by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200% or more, relative to normal cell surface expression of a PSMA-expressing cell or relative to cell surface expression in the absence of such compound. For example, an antiandrogen may, in some embodiments, increase the cell surface expression of PSMA on cancer cells by at least 20%, or more.

A composition, in some embodiments, includes a physiologically or pharmaceutically acceptable carrier, excipient, or stabilizer combined with a compound that increases cell surface expression of PSMA and/or aPSMA ligand conjugate. As used herein, “pharmaceutically acceptable carrier” or “physiologically acceptable carrier” includes any and all salts, solvents, dispersion media, coatings, antibacterial and antifungal agents, isotonic and absorption delaying agents, and the like that are physiologically compatible. A “pharmaceutically-acceptable carrier,” as used herein, refers to one or more compatible solid or liquid fillers, diluents or encapsulating substances that are suitable for administration into a human. The term “carrier” denotes an organic or inorganic ingredient, natural or synthetic, with which the active ingredient is combined to facilitate the application. A carrier may be suitable for intravenous, intramuscular, subcutaneous, parenteral, spinal or epidermal administration (e.g., by injection or infusion).

In some embodiments, a composition may be administered to a subject in pharmaceutically-acceptable amounts and in pharmaceutically-acceptable compositions. The term “pharmaceutically acceptable” means a non-toxic material that does not interfere with the effectiveness of the biological activity of the active ingredients (e.g., PSMA ligands, anticancer agents, cytotoxic agents, antiandrogens, mTOR inhibitors). Such compositions may contain salts, buffering agents, preservatives, compatible carriers, and optionally other therapeutic agents, such as supplementary immune potentiating agents including adjuvants, chemokines and cytokines. When used in medicine, the salts should be pharmaceutically acceptable, but non-pharmaceutically acceptable salts may conveniently be used to prepare pharmaceutically-acceptable salts thereof and are not excluded.

A salt retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The pharmaceutical compositions may contain suitable buffering agents, including: acetic acid in a salt; citric acid in a salt; boric acid in a salt; and phosphoric acid in a salt.

In some embodiments, a composition may contain suitable preservatives, such as: benzalkonium chloride; chlorobutanol; parabens and/or thimerosal.

In some embodiments, a composition may conveniently be presented in unit dosage form and may be prepared by any of the methods well-known in the art of pharmacy. All methods include the step of bringing the active compound(s) (e.g., PSMA ligand, anticancer agent, cytotoxic agent and/or compound that upregulates cell surface expression of PSMA) into association with a carrier that constitutes one or more accessory ingredients. In some embodiments, compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous or non-aqueous preparation of PSMA ligand conjugates and/or compounds that increase cell surface expression of PSMA (e.g., antiandrogens, mTOR inhibitors), which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulations suitable for oral, subcutaneous, intravenous, intramuscular, etc. administration can be found in Remington's Pharmaceutical Sciences, Mack Publishing Co., Easton, Pa. Any of the compositions provided herein may be sterile.

Active compounds (e.g., PSMA ligand, anticancer agent, cytotoxic agent and/or compound that upregulates cell surface expression of PSMA) can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

A composition can be administered by any conventional route, including injection or by gradual infusion over time. The administration may, for example, be oral, intravenous, intraperitoneal, intramuscular, intracavity, intratumor, or transdermal. When compositions are used therapeutically, preferred routes of administration include intravenous and by pulmonary aerosol. Techniques for preparing aerosol delivery systems containing antibodies are well known to those of skill in the art. Generally, such systems should utilize components that will not significantly impair the biological properties of the antibodies, such as the paratope binding capacity (see, for example, Sciarra and Cutie, “Aerosols,” in Remington's Pharmaceutical Sciences, 18th edition, 1990, pp. 1694-1712; incorporated by reference). Those of skill in the art can readily determine the various parameters and conditions for producing antibody aerosols without resorting to undue experimentation.

Compositions as provided herein, in some embodiments, may be administered in effective amounts. An “effective amount” is that amount of an active compound (e.g., PSMA ligand conjugate and/or compound that increases cell surface expression of PSMA) that alone, or together with further doses, produces the desired response, e.g., inhibits cell proliferation of PSMA-expressing cells and/or kills PSMA-expressing cells. For cancer, this may involve only slowing the progression of a cancer, for example, temporarily, although more preferably, it involves halting the progression of the cancer permanently. This can be monitored by routine methods. The desired response to treatment of cancer or other disease or condition also can be delaying the onset or even preventing the onset of the cancer or other disease or condition.

Such effective amounts will depend, of course, on the particular condition being treated (e.g., PSMA-expressing cancer), the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient/subject may insist upon a lower dose or tolerable dose for medical reasons, psychological reasons or for virtually any other reason.

Compositions as provided herein, in some embodiments, are sterile and contain an effective amount of PSMA ligand conjugates and/or compound that increases cell surface expression of PSMA, etc. for producing the desired response in a unit of weight or volume suitable for administration to a patient/subject. The response can, for example, be measured by determining the physiological effects of the composition, such as regression of a tumor or decrease of disease symptoms. Other assays will be known to one of ordinary skill in the art and can be employed for measuring the level of the response.

The doses of compositions administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

The synergistic effect provided by various combinations of PSMA ligand conjugates with compounds provided herein, such as antiandrogens or mTOR inhibitors, may result in efficacious doses which are lower than the doses required for efficacy when either component of the combination is used singly. The added advantage of a dose lowering effect may have the added benefit of a wider safety profile, i.e., fewer adverse side effects.

For example, doses of PSMA ligand conjugate and/or compound that increases cell surface expression of PSMA can range from about 10 μg/kg to about 100,000 μg/kg. Based on the composition, the dose can be delivered continuously, such as by continuous pump, or at periodic intervals. Desired time intervals of multiple doses of a particular composition can be determined without undue experimentation by one skilled in the art. Other protocols for the administration of compositions will be known to one of ordinary skill in the art, in which the dose amount, schedule of administration, sites of administration, mode of administration and the like vary from the foregoing.

In some embodiments, the dose of PSMA ligand conjugate is administered intravenously. In such embodiments, the dose of PSMA ligand conjugate may be about 1.0 mg/kg to 2.5 mg/kg. For example, in some embodiments, the dose of PSMA ligand conjugate administered intravenously is 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, or 2.5 mg/kg. In some embodiments, the dose of PSMA ligand administered intravenously conjugate is about 1.0 mg/kg to 2.3 mg/kg. In an embodiment, the PSMA ligand conjugated is a PSMA ADC, and the PSMA ADC is provided in a dose of about 1.8 mg/kg to 2.3 mg/kg.

The length of time during which a PSMA ligand conjugate is administered administered intravenously may vary. In some embodiments, a PSMA ligand conjugate may be administered intravenously for 30 minutes, 35 minutes, 40 minutes, 45 minutes, 50 minutes, 55 minutes, 60 minutes, 65 minutes, 70 minutes, 75 minutes, 80 minutes, 85 minutes or 90 minutes.

In some embodiments, a PSMA ligand conjugate is administered intravenously at repeated intervals such as, for example, once a week, once every two weeks, or once every three weeks for up to a total of four, six or eight doses. In some embodiments, the PSMA ligand conjugate is administered intravenously twice a week, or more.

In some embodiments, a PSMA ligand conjugate may be administered intravenously for about 60 minutes once every three weeks at a dose of about 1.0 mg/kg to 2.3 mg/kg for up to a total of eight doses.

In some embodiments, a compound as provided herein, such as an antiandrogen, is administered in the form of oral capsules. In such embodiments, the dose of such compound may be about 40 mg to about 160 mg (e.g., 20 mg or 40 mg per capsule). For example, in some embodiments, the dose of such compound administered orally is 40 mg, 60 mg, 80 mg, 100 mg, 120 mg, 140 mg or 160 mg. In some embodiments, the oral dose of such compound is administered once daily, or twice daily, for about 21 to about 28 days. In some embodiments, the dose of such compound is 80 mg (e.g., 2 oral capsules, 40 mg each) to 160 mg (e.g., 4 oral capsules, 40 mg each) and is administered once daily (OD) for 28 days. In some embodiments, such compound is enzulatamide, at a dose of 80 mg (e.g., 2 oral capsules, 40 mg each) to 160 mg (e.g., 4 oral capsules, 40 mg each) and is administered once daily (OD) for 28 days.

In some embodiments, the oral dose of a compound as provided herein, such as an antiandrogen, may be about 500 mg to about 1000 mg. For example, in some embodiments, the dose of such compound administered orally is 500 mg, 550 mg, 600 mg, 650 mg, 70 mg 0, 750 mg, 800 mg, 850 mg, 900 mg, 950 mg or 1000 mg. In some embodiments, the oral dose of such compound is administered once daily, or twice daily, for about 21 to about 28 days. In some embodiments, the dose of such compound is 500 mg to 1000 and is administered once daily (OD) for 28 days. In some embodiments, such compound is abiraterone or abiraterone acetate, at a dose of 500 mg to 1000 mg, and is administered once daily (OD) for 28 days.

In some embodiments, an mTOR inhibitor is administered in an I.V. dose of about 5 to 25 mg at weekly intervals. In another embodiment, the mTOR inhibitor is administered in an I.V. dose of 5 to 10 mg at weekly intervals. In one embodiment, the mTOR inhibitor is temsirolimus (Toresal®) dosed 25 mg I.V. over 30 to 60 minutes at weekly intervals.

In general, doses of radionuclides delivered by a PSMA ligand conjugate provided herein can range from about 0.01 mCi/Kg to about 10 mCi/kg. In some embodiments, the dose of radionuclide ranges from about 0.1 mCi/Kg to about 1.0 mCi/kg. In one embodiment, the PSMA ligand conjugate is ¹³¹I-MIP-1095 provided in an I.V. dose of about 1 to 10 GBq. In another embodiment, ¹³¹I-MIP-1095 is provided in a dose range of 2 to 8 GBq. In yet another embodiment, the mean dose is about 5 GBq. The optimal dose of a given isotope can be determined empirically by simple routine titration experiments well known to one of ordinary skill in the art.

Administration of compositions provided herein to mammals other than humans, e.g. for testing purposes or veterinary therapeutic purposes, is carried out under substantially the same conditions as described above.

Compositions (e.g., that comprise PSMA ligand conjugate and/or compound that increases cell surface expression of PSMA) as provided herein have in vitro and in vivo diagnostic and therapeutic utilities. For example, these compounds can be administered to cells in culture, e.g., in vitro or ex vivo, or in a subject, e.g., in vivo, to treat, prevent or diagnose cancer or other disease or condition. As used herein, the term “subject” is intended to include humans and non-human animals. Preferred subjects include a human patient having a disorder characterized by expression, typically aberrant expression (e.g., overexpression) of PSMA.

The compositions provided herein, in some embodiments, may be used in conjunction with other therapeutic treatment modalities. Such other treatments include surgery, radiation, cryosurgery, thermotherapy, hormone treatment, chemotherapy, vaccines, and other immunotherapies.

Subjects with prostate cancer, in some embodiments, have undergone, are undergoing, or will undergo hormone therapy. Thus, in some embodiments, the compositions provided herein may be administered to a subject subsequent to, together with, or prior to hormone therapy, such as for prostate cancer. Examples of hormone therapies for prostate cancer include, without limitation: luteinizing hormone-releasing hormone agonists (e.g., leuprolide, goserelin, and buserelin), which can stop the testicles from making testosterone; antiandrogens (e.g., flutamide, bicalutamide, enzalutamide and nilutamide), as discussed elsewhere herein, which can block the action of androgens such as testosterone; drugs that can prevent the adrenal glands from making androgens (e.g., ketoconazole and aminoglutethimide); orchiectomy, which is a surgical procedure to remove one or both testicles, the main source of male hormones such as testosterone, to decrease the amount of hormone being produced; and estrogens, which can prevent the testicles from making testosterone.

A myriad of subjects may benefit from the methods and compositions provided herein. In some embodiments, such a subject has progressive metastatic castration-resistant prostate cancer despite a castrate level of serum testosterone (e.g., <50 mg/dL) and having had prior chemotherapy with docetaxel. In other embodiments, the subject has metastatic castration resistant prostate cancer, has had prior treatment with taxane chemotherapy and has received and progressed on abiraterone and/or enzalutamide. In yet other embodiments, the subject has progressive metastatic castration-resistant prostate cancer despite a castrate level of serum testosterone, has had prior treatment with abiraterone and/or enzalutamide, and has had no prior treatment with cytotoxic chemotherapy. In still other embodiments, the subject has progressive metastatic castration-resistant prostate cancer despite a castrate level of serum testosterone and has had one prior treatment with abiraterone and/or enzalutamide. In further embodiments, the subject has progressive metastatic castration-resistant prostate cancer despite a castrate level of serum testosterone and has had no prior treatment with abiraterone and or enzalutamide. In additional embodiments, the subject has asymptomatic or minimally symptomatic metastatic castration-resistant prostate cancer despite a castrate level of serum testosterone and has had no prior treatment with abiraterone and/or enzalutamide. In some embodiments, the subject has stable metastatic castration-resistant prostate cancer and is receiving treatment with abiraterone and/or enzalutamide. In other embodiments, the subject has biochemically recurrent prostate cancer and has previously undergone a primary therapy (e.g., radical prostatectomy (e.g., open, laparoscopic, or robot-assisted) or radiation therapy (e.g., dose-escalated three-dimensional conformal RT, intensity-modulated RT, brachytherapy, or a combination thereof)). In yet other embodiments, the subject has localized high-risk prostate cancer (e.g., prostate specific antigen (PSA) greater than 10 nanogram per milliliter (ng/ml); PSA velocity greater than 2 ng/ml per/year (defined as a rise in PSA of greater than 2 ng/ml in the preceding 12 month period); Gleason score greater than or equal to 7 (4+3); or Gleason score 6 if either PSA greater than or equal to 10 ng/ml or PSA velocity greater than or equal to 2 ng/ml/year) and is a candidate for prostatectomy.

Also provided herein are kits comprising the composition(s). In some embodiments, the kits comprise a container containing a compound that increases cell surface expression of PSMA, and a container containing a PSMA ligand conjugate (or the components thereof). The kits can further contain at least one additional reagent as provided herein. In some embodiments, a kit may comprise a carrier being compartmentalized to receive in close confinement therein one or more containers or series of containers such as test tubes, vials, flasks, bottles, syringes, or the like. One container or series of containers may contain one or more compound that increases cell surface expression of PSMA. Another container, or series of containers in some embodiments, may contain a PSMA ligand conjugate (or the components thereof). The components of the kits can be packaged either in aqueous medium or in lyophilized form. The components of the conjugates can be supplied either in fully conjugated form, in the form of intermediates or as separate moieties to be conjugated by the user of the kit.

Kits may, in some embodiments, also comprise a diluent and/or instructions for reconstituting lyophilized forms of the PSMA ligand conjugates and/or compounds for increasing cell surface expression of PSMA, or instructions for diluting aqueous components of the kits. Kits may also comprise instructions for combining a compound that increases cell surface expression of PSMA and/or a PSMA ligand conjugate.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference. However, the citation of any reference is not intended to be admission that said reference is prior art.

### EXAMPLES

### Example 1

### PSMA ADC Synergizes with Antiandrogens and Rapamycin

Inhibitors were tested for anti-proliferative activity individually and in combination across a range of concentrations in a matrix fashion Inhibition data were compared with Bliss predictions, and the differences between the experimental and predicted results were calculated for each point in the matrix. Positive differences indicate supra-additive or synergistic effects. Negative differences indicate antagonism. Results were assessed for statistical significance, as described below.

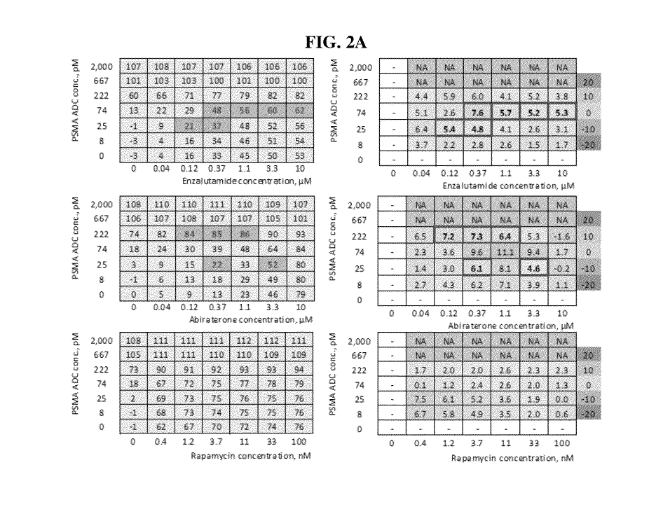

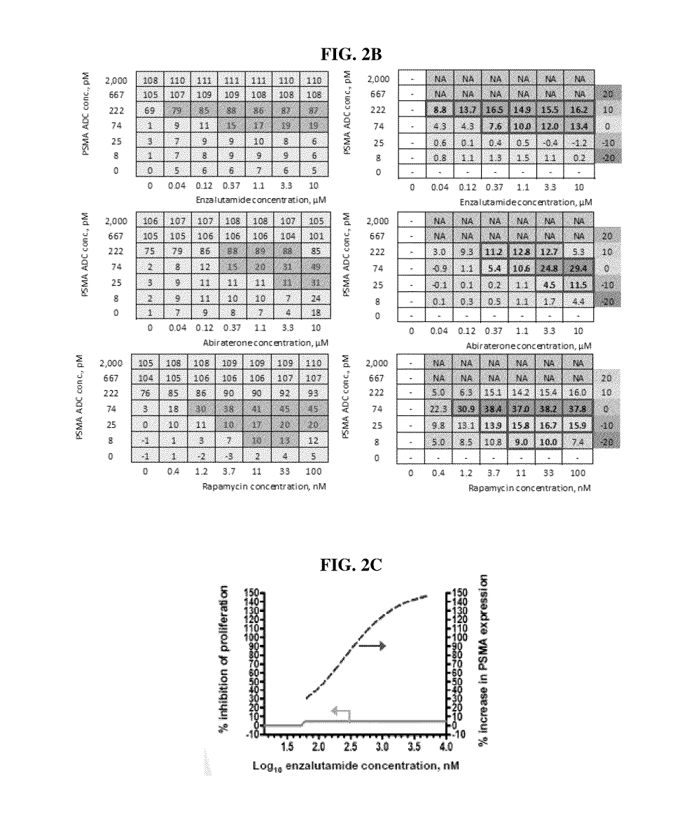

Mean inhibition data and Bliss differences are shown in FIG. 2 for PSMA antibody-drug conjugate (PSMA ADC) combined with enzalutamide, abiraterone or rapamycin. Bliss differences are depicted via heat maps of values that are positive (medium gray), negative (dark gray), or near zero (light gray). Boxes are used to indicate statistically significant Bliss differences and the corresponding percent inhibitions. PSMA ADC exhibited similar single-agent activity against LNCaP and C4-2 cells, with IC₅₀ values of approximately 200 pM in each case. These results are in line with prior reports (20, 21). Enzalutamide, abiraterone, and rapamycin each inhibited proliferation of LNCaP cells (FIG. 2A) but had minimal antiproliferative effects on C4-2 cells when used as single agents (FIG. 2B).

Despite lack of direct antiproliferative activity in C4-2 cells, enzalutamide, abiraterone and rapamycin all significantly enhanced the activity of PSMA ADC. Bliss differences ranged to nearly 40% and were statistically significant across a range of inhibitor concentrations and levels of inhibition (FIG. 2B). No significant antagonism was observed for these combinations under any condition. Statistically significant synergy also was observed for PSMA ADC/enzalutamide and PSMA ADC/abiraterone combinations in LNCaP cells (FIG. 2A); however, the breadth and magnitude were more limited as compared to C4-2 cells. Similarly, Bliss differences were generally positive across the matrix of concentrations for the PSMA ADC/rapamycin combination in LNCaP cells; however, no value reached statistical significance.

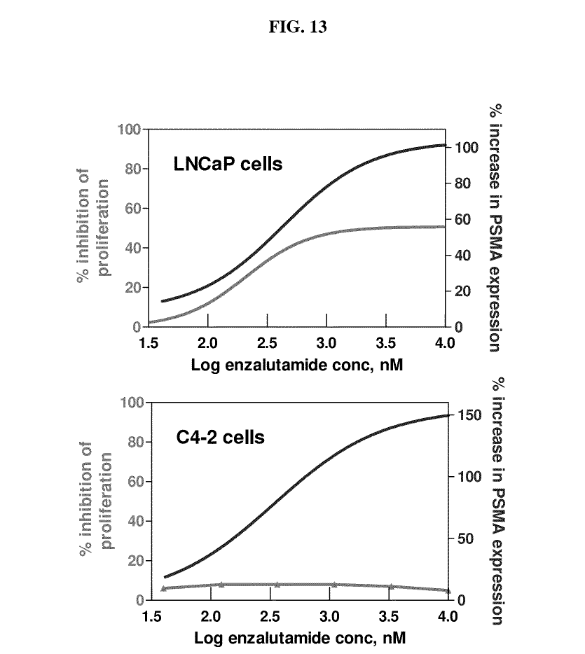

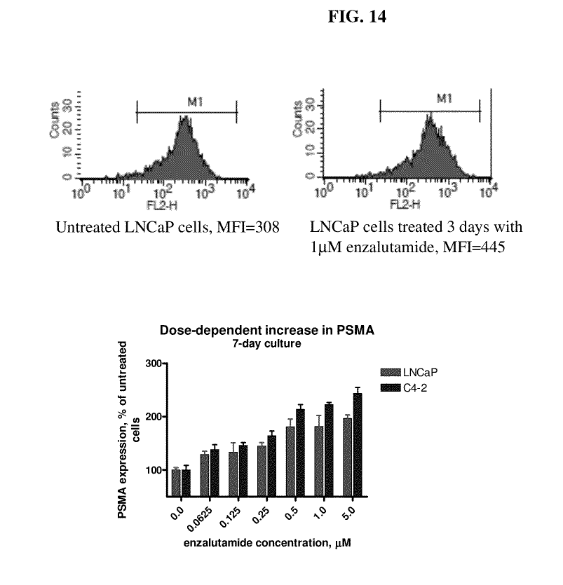

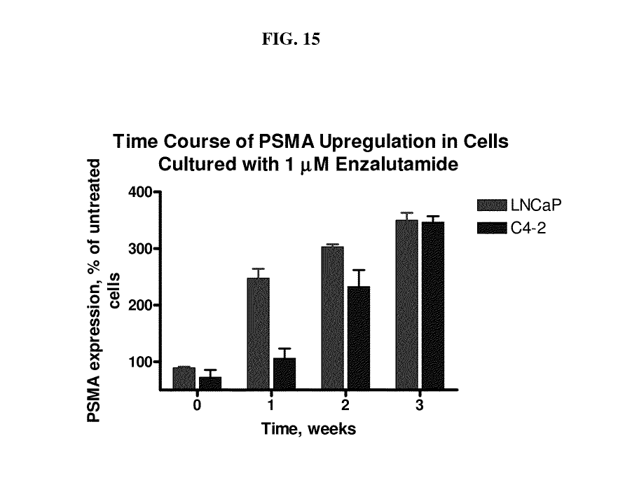

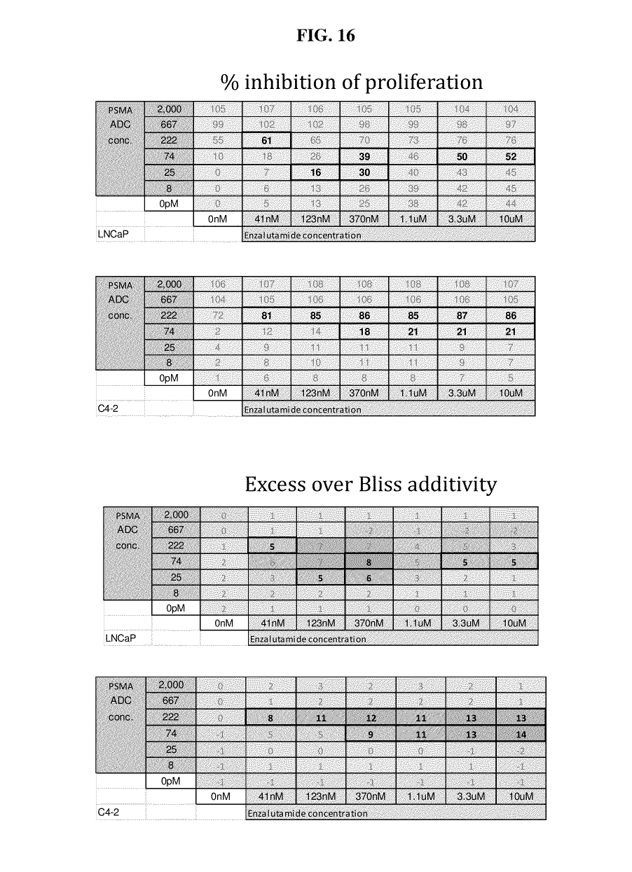

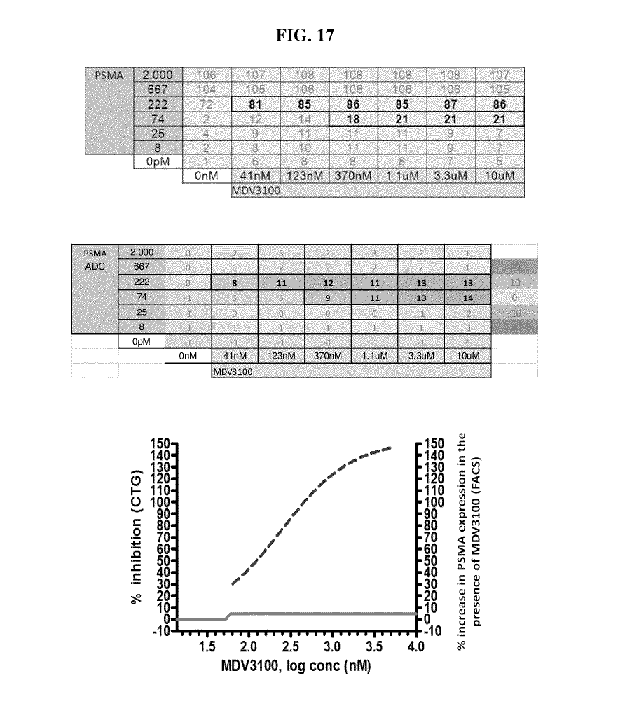

Mean inhibition data and Bliss differences are shown in FIG. 13 for PSMA ADC combined with the antiandrogens, ARN-509 or TOK-001. Bliss differences are depicted via heat maps of values that are positive (medium gray), negative (dark gray), or near zero (light gray). ARN-509 and TOK-001 each inhibited proliferation of LNCaP cells but had minimal antiproliferative effects on C4-2 cells when used as single agents. Despite lack of direct antiproliferative activity in C4-2 cells, ARN-509 and TOK-001 enhanced the activity of PSMA ADC.

To determine if the observed synergies were unique to PSMA ADC/antiandrogen combinations, a small molecule ligand was tested in combination with antiandrogens. Mean inhibition data and Bliss differences are shown in FIG. 10 for the small molecule PSMA ligand-anticancer agent conjugate, EC1069, combined with enzalutamide. Heat maps illustrate Bliss differences that are positive for synergy (medium gray), negative (dark gray), or near zero (light gray).

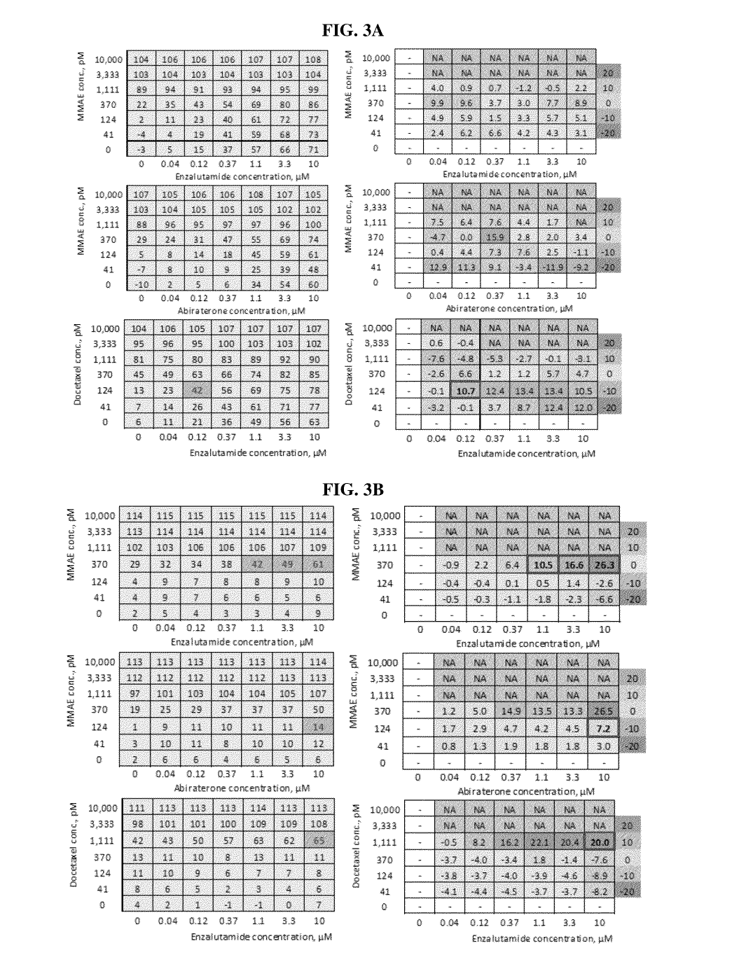

As an initial step towards dissecting mechanisms of synergy, the antiandrogens and rapamycin were combined with free MMAE and with unmodified PSMA mAb. Free MMAE showed subnanomolar potency (IC₅₀˜0.5 nM) against both LNCaP and C4-2 cells, as expected (21). When paired with enzalutamide or abiraterone, free MMAE exhibited additive activity in LNCaP cells and additive to weakly synergistic activity on C4-2 (FIG. 3). Similar results were obtained when docetaxel, another microtubule inhibitor, was combined with enzalutamide. There were only sporadic instances of statistically significant synergy for the docetaxel/enzalutamide combination (FIG. 3).

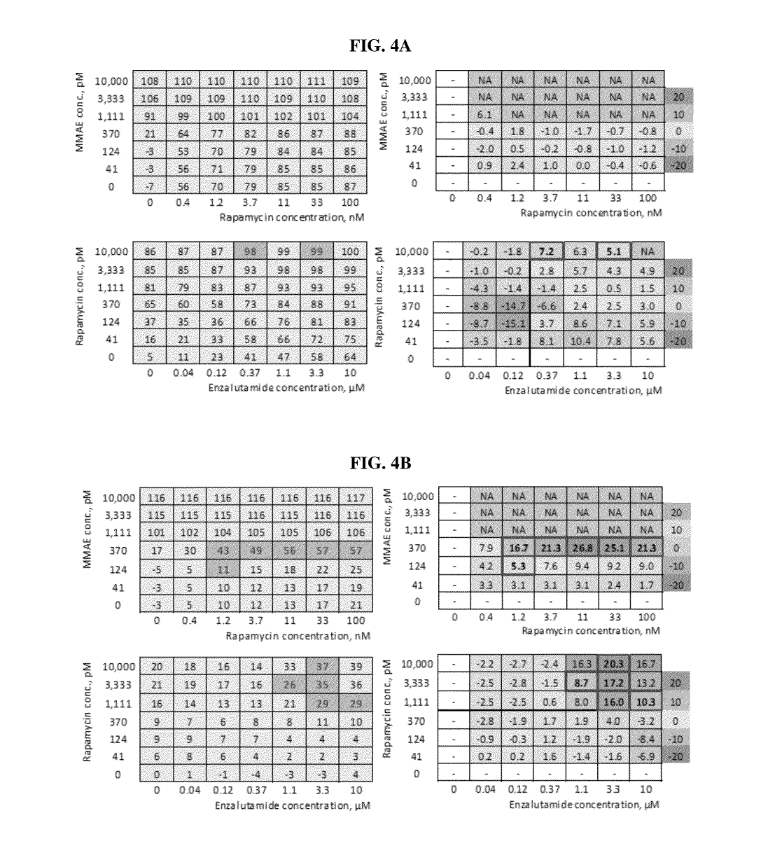

The rapamycin/MMAE combination showed additive effects in LNCaP and moderate synergy in C4-2 cells (FIG. 4). Thus, rapamycin synergized less potently with MMAE as compared with PSMA ADC on both cell lines. Nonetheless, the rapamycin/MMAE combination appeared to be as or more potent than the enzalutamide/MMAE or abiraterone/MMAE combination in C4-2 cells. Weak to moderate synergy was observed between rapamycin and enzalutamide in both cell lines. In C4-2 cells, non-inhibitory concentrations of enzalutamide enhanced the weak antiproliferative activity of rapamycin (FIG. 4).

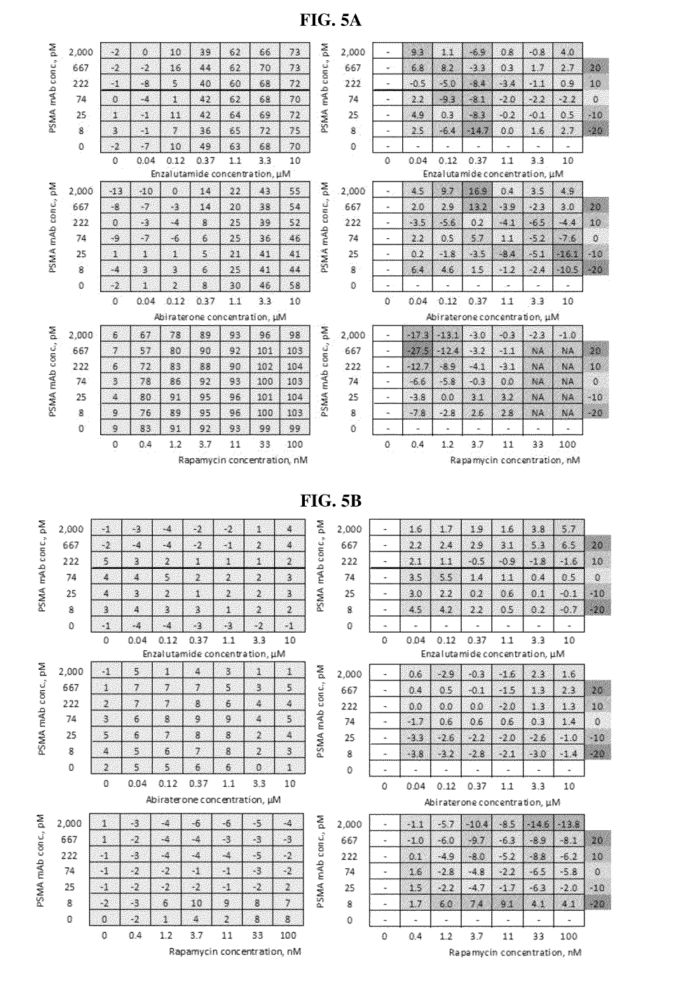

Unmodified PSMA mAb was inactive both alone and in combination with enzalutamide, abiraterone or rapamycin (FIG. 5). Due to the very limited activity of the individual agents or combinations, assays were performed only twice in some cases. Thus, the robust synergies observed upon combining PSMA ADC with antiandrogens are specific to the conjugate and are not reproduced with either of its individual components.

As a control, a mock combination was prepared and assessed for synergy by adding PSMA ADC to the assay plate via two separate additions. As expected, the mock combination did not show statistically significant synergy or antagonism in either cell line.

### Example 2

### Antiandrogens Reversibly Increase PSMA Expression in a Time- and Dose-Dependent Manner

Additional studies examined treatment-induced changes in PSMA expression as a potential correlate of synergy. Flow cytometry and Western blotting were used to assess cell-surface and total PSMA, respectively.

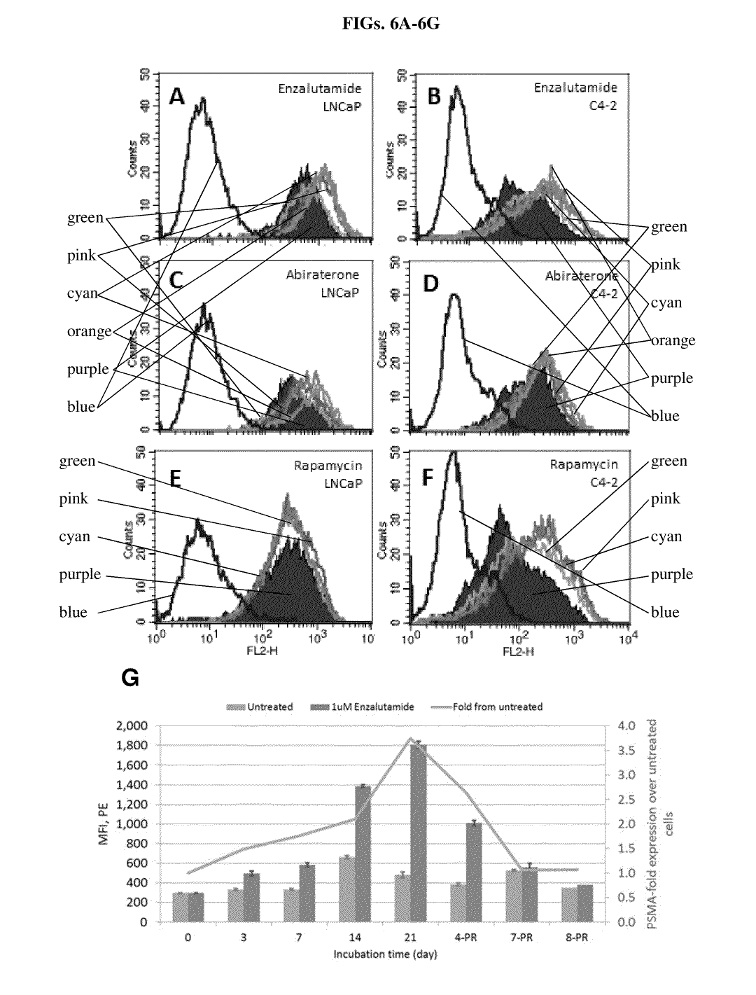

Enzalutamide and abiraterone increased cell-surface levels of PSMA in a dose-dependent manner in both cell lines (FIG. 6A-D). PSMA expression was approximately doubled at antiandrogens concentrations above 1 mM at 7 days post-treatment. In contrast, rapamycin increased PSMA expression in C4-2 cells only (FIG. 6E-F). Low nanomolar concentrations of rapamycin were sufficient to induce a >2-fold increase in PSMA expression in C4-2 cells.

To probe the dynamics of expression, C4-2 cells were cultured in enzalutamide (1 mM) for 3 weeks prior to continued culture in the absence of drug for an additional 8 days. Cell-surface PSMA was measured by flow cytometry. PSMA expression increased steadily over time in the presence of enzalutamide. After 21 days, PSMA expression on treated cells was nearly 4-fold greater than expression on untreated cells passaged in parallel. PSMA expression returned to baseline levels within 7 days of culture in the absence of enzalutamide (FIG. 6G).

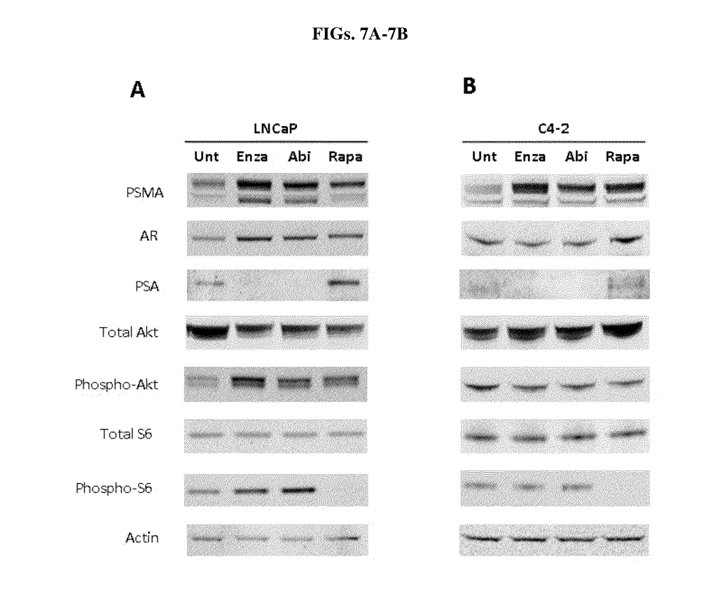

Treatment-induced increases in PSMA were also apparent by Western blotting (FIG. 7). The magnitude of increase was approximately 5-fold for enzalutamide-treated LNCaP cells as determined by serial dilution of lysates and semi-quantitative analysis of the Western blots (data not shown). Enzalutamide, abiraterone and rapamycin upregulated PSMA to similar extents in C4-2 cells. The magnitude of PSMA expression in the presence of the antiandrogens approached but seldom exceeded the expression seen in cells cultured in charcoal-stripped serum. Overall, the flow cytometry and Western blot data showed consistent patterns of treatment-induced changes in PSMA expression.

### Example 3

### Treatment-Induced Changes in AR, PSA and PI3K Pathway Components

Levels of AR, PSA, total Akt, phospho-Akt (S473), total S6, and phospho-S6 were also evaluated pre- and post-treatment by Western blotting. Actin was probed as a measure of total cell loading. As expected, antiandrogen-induced upregulation of PSMA was accompanied by downregulation of PSA (FIG. 7). In contrast, rapamycin increased PSA expression. This result is consistent with prior observations for rapamycin or its analogs (28, 29). The co-upregulation of PSMA and PSA by rapamycin contrasts with the opposing effects on PSMA and PSA exerted by antiandrogens. AR protein levels were affected modestly by treatment (FIG. 7).

Akt activation was a common response to antiandrogen or rapamycin treatment of LNCaP cells. However, treatment had limited effects on Akt activation in C4-2 cells. Similarly, the antiandrogens increased levels of phospho-S6 in LNCaP but not C4-2 cells. These findings are consistent with an mTOR-mediated adaptation to antiandrogen treatment in LNCaP cells. Rapamycin-mediated activation of Akt is known to occur via disruption of a negative feedback loop involving insulin receptor substrate 1 (30-33). As expected, rapamycin effectively ablated S6 phosphorylation in both LNCaP and C4-2, indicating that rapamycin is pharmacologically active in both cell lines (FIG. 7).

### Example 5

### Additive to Weakly Synergistic Effects for Other Drug Combinations

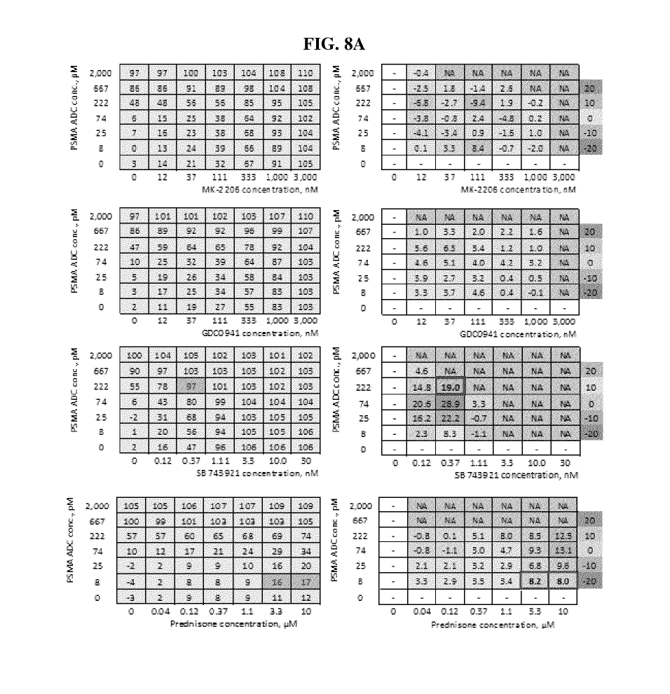

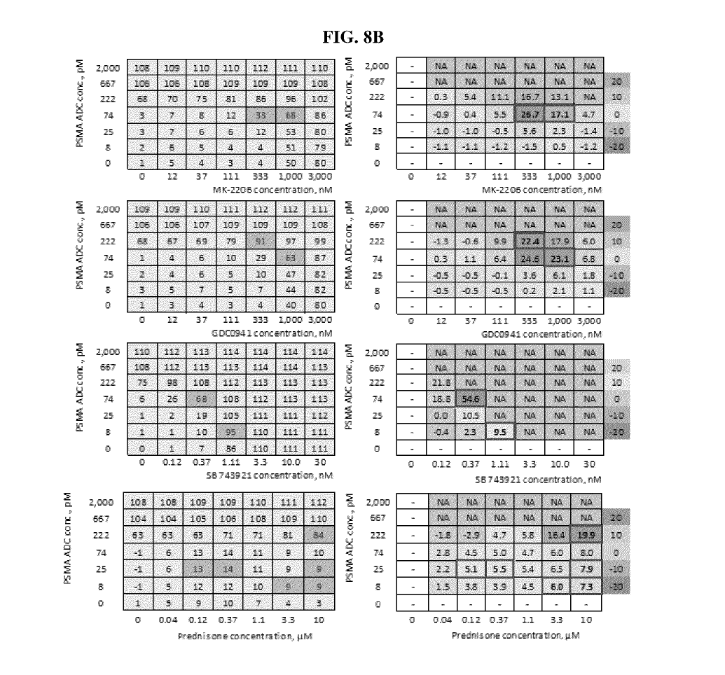

In exploratory studies, an additional group of compounds was screened for activity alone and in combination with PSMA ADC. These agents include the Akt inhibitor MK-2206 (34); the PI3K inhibitor GDC-0941 (35); prednisone; and SB-743921 (36), an inhibitor of the kinesin spindle protein (ksp) Eg5. MK-2206 and GDC-0941 are selective inhibitors of upstream components of the PI3K/mTOR pathway. Prednisone is a component of abiraterone- and docetaxel-based treatment regimens in prostate cancer. SB-743931 was included in our study based on gene expression profiling within the cBio cancer genomics database (37), which revealed coordinated expression (P=0.000004) of the genes for PSMA and Eg5 within primary and metastatic prostate cancer tumors. Each of these compounds showed additive to weakly synergistic activity when combined with PSMA ADC (FIG. 8). That is, Bliss differences trended towards synergy for most conditions and reached statistical significance in a few isolated cases. No significant antagonism was observed for any combination. Similar results were obtained when prednisone was replaced with its active metabolite, prednisolone.

Table 2 provides an overview of the synergies observed in this study. For each drug:drug combination, the breadth of synergy is represented as the percentage of evaluable conditions that resulted in statistically significant synergy across the matrix of drug concentrations. Combinations are sorted, highest to lowest, according to the breadth of synergy observed in LNCaP cells. The PSMA ADC/enzalutamide and PSMA ADC/abiraterone combinations showed the broadest synergy in LNCaP cells. Next were PSMA ADC combinations with prednisone or SB-743921. For the PSMA ADC/SB-743921 combination, the data should be interpreted with caution, since several drug-drug combinations resulted in >100% inhibition and thus were not evaluable for Bliss differences. Further testing of this combination over a more focused range of concentrations is warranted. Most combinations did not show significant synergy in LNCaP cells.

TABLE 2
Summary of synergies observed for different drug combinations.

| | LNCaP | | | C4-2 | | |
|---|---|---|---|---|---|---|
| | Number of | Number of | | Number of | Number of | |
| | synergistic | evaluable | | synergistic | evaluable | |
| | conditions in | conditions in | % | conditions in | conditions in | % |
| | drug:drug | drug:drug | synergistic | drug:drug | drug:drug | synergistic |
| Combination | matrix | matrix | conditions | matrix | matrix | conditions |
| PSMA ADC + Enzalutamide | 6 | 24 | 25.0 | 10 | 24 | 41.7 |
| PSMA ADC + Abiraterone | 5 | 24 | 20.8 | 9 | 24 | 37.5 |
| PSMA ADC + Prednisone | 2 | 24 | 8.3 | 6 | 24 | 25.0 |
| PSMA ADC + SB 743921 | 1 | 12 | 8.3 | 2 | 8 | 25.0 |
| Rapamycin + Enzalutamide | 2 | 35 | 5.7 | 5 | 36 | 13.9 |
| Docetaxel + Enzalutamide | 1 | 26 | 3.8 | 1 | 24 | 4.2 |
| PSMA ADC + Rapamycin | 0 | 24 | 0.0 | 11 | 24 | 45.8 |
| MMAE + Rapamycin | 0 | 19 | 0.0 | 6 | 18 | 33.3 |
| MMAE + Enzalutamide | 0 | 24 | 0.0 | 3 | 18 | 16.7 |
| PSMA ADC + MK-2206 | 0 | 25 | 0.0 | 2 | 23 | 8.7 |
| PSMA ADC + GDC0941 | 0 | 25 | 0.0 | 2 | 24 | 8.3 |
| MMAE + Abiraterone | 0 | 23 | 0.0 | 1 | 18 | 5.6 |
| PSMA mAb + Abiraterone | 0 | 36 | 0.0 | 0 | 36 | 0.0 |
| PSMA mAb + Enzalutamide | 0 | 36 | 0.0 | 0 | 36 | 0.0 |
| PSMA mAb + Rapamycin | 0 | 26 | 0.0 | 0 | 36 | 0.0 |
| PSMA ADC + PSMA ADC | 0 | 24 | 0.0 | 0 | 15 | 0.0 |

Synergy was more widespread in C4-2 cells. The PSMA ADC/enzalutamide and PSMA ADC/abiraterone combinations exhibited a high frequency of synergistic conditions in C4-2 cells, as did rapamycin in combinations with PSMA ADC or MMAE. Other combinations (e.g., PSMA ADC in combination with MK-2206 or GDC0941) also exhibited significant synergy in C4-2 but not LNCaP cells (Table 2). Combinations that did not show significant synergy in either LNCaP or C4-2 cells were those that involved PSMA mAb and the PSMA ADC mock combination.

Materials and Methods

Materials

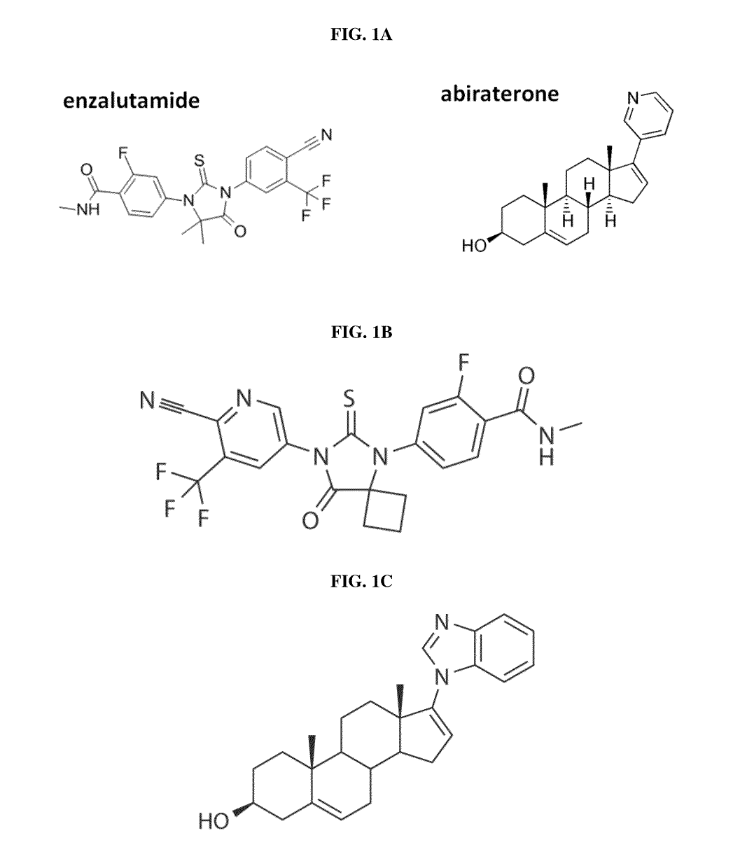

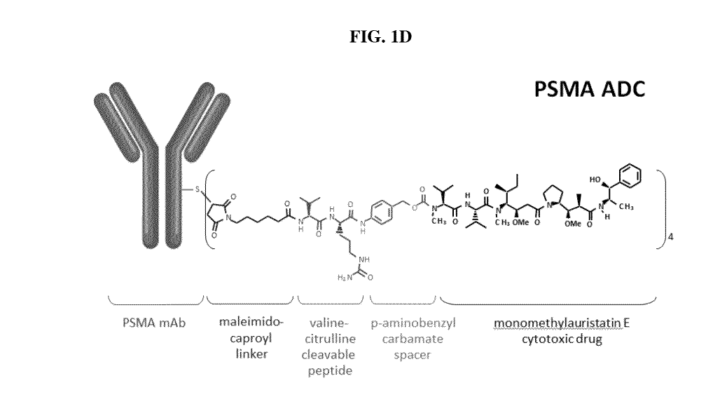

LNCaP cells were obtained from American Type Culture Collection (ATCC). C4-2 is an androgen-independent subclone of LNCaP (22). LNCaP and C4-2 are characterized by a T877A mutation in AR and loss of PTEN expression (23, 24). The cell lines allow investigations of androgen dependence in an isogenic background. Both cell lines were passaged in RPMI1640 (Mediatech Inc.) supplemented with 2 mM L-glutamine, 10 mM HEPES, 1 mM sodium pyruvate, 100 μM non-essential amino acids, 1% penicillin/streptomycin and 10% fetal bovine serum (FBS, Life Technologies). Cells were drawn from a common bank and then used within 10 passages. PSMA ADC was prepared as described (20), and enzalutamide and abiraterone (parent drug) were obtained from MedKoo Biosciences (FIG. 1). Prednisone and prednisolone were purchased from Sigma; rapamycin was from EMD Millipore. GDC0941, MK-226 and SB-743921 were from Selleck Chemicals. The following antibodies were obtained from Santa Cruz Biotechnology: Anti-AR (sc-7305, 441) and anti-PSA (sc-7638, C-19). Two murine anti-PSMA mAbs were used, MAB544 (Maine Biotechnology) for Westerns and 3.9 (Progenics) for flow cytometry. The following antibodies were obtained from Cell Signaling: Total Akt (#2920S), phospho-Akt (S473, #4051L), total-S6 (#2317S), and phospho-S6 (#2211L). Anti-actin antibody (MAB1501) was from Millipore. Isotype-specific secondary IRDye antibodies were from LI-COR Biosciences.

Cell Viability Assay

Cells were plated at a density of 1×10³ cells in 25 μL in 384-well white-colored microplates (Perkin Elmer) and cultured overnight. The next day, cells were treated with 1 or 2 drugs in a total volume of 50 μL. Cultures were incubated at 37° C. for 7 days. Viability was assessed on days 0 and 7 using CellTiter-Glo (Promega) and an Analyst GT luminescence reader at 560 nm emission (Molecular Devices). The percent inhibition of proliferation was calculated as (Vᵤ−Vₜ)/(Vᵤ−V₀)×100, where Vᵤ, Vₜ and V₀ represent the viability values for untreated cells on day 7, treated cells on day 7, and cells prior to treatment on day 0, respectively. A value of 100% reflects complete inhibition of proliferation (Vₜ=V₀). Values of >100% reflect cytotoxic compounds or combinations, where Vₜ<V₀.

Flow Cytometry

For short-term treatments (≦7 days), cells were plated on 24-well plates (BD Falcon) overnight, at a density of 200,000 cells per 500 μL. The next day, wells were exchanged with 500 μL of fresh media that included inhibitor, and incubated at 37° C. until the indicated time point. For the long-term treatments (>7 days), cells were plated on T-25 flasks (BD Falcon) overnight, at a density of 500,000 cells per flask in 5 mL of media. 5 mL of fresh media with or without treatment was exchanged the next day. On weekly splits thereafter, cells were detached with Cell Dissociation solution (Sigma), washed with fresh media, counted using Vi-CELL XR (Beckman Coulter), and split into two T-25 flasks for continued culture with or without inhibitor. Prior to analysis, cells were detached, counted and suspended in PBS (PBS(−), without Ca/Mg, Life Technologies) containing 0.3% bovine serum albumin (BSA, Sigma) and 0.1% sodium azide (VWR). Cells (100,000 in 100 μL) were placed in round-bottom 96-well plates (Falcon) and incubated for 30 minutes at room temperature with 1 μg/mL of anti-PSMA mAb 3.9 conjugated to phycoerythrin (PE). Cells then were washed twice with 200 μL PBS/BSA/azide buffer and read using a FACSCalibur instrument (BD Biosciences). A mouse IgG2b-PE conjugate of irrelevant specificity (Abcam) was included as an isotype control.

Western Blotting

Cells (300,000) were incubated overnight in 6-well plates (BD Falcon) in 4 mL of 10% FBS RPMI1640 media prior to addition of inhibitors. Plates were incubated at 37° C. for an additional 7 days, washed once with 5 mL of cold PBS(−) and placed on ice. Cells were lysed with 130 μL of RIPA Lysis Buffer (Santa Cruz Biotechnology) and then centrifuged at 14,000×g for 10 minutes at 4° C. The supernatant was quantitated for protein concentration using a BCA kit (Pierce/Thermo), resolved under reducing conditions using NuPAGE Novex 4-12% Bis-Tris gels (Life Technologies), and transferred to nitrocellulose using the iBlot 7-Minute Blotting System (Life Technologies). Membranes were blocked using Odyssey Blocking Reagent (LI-COR Biosciences), incubated with primary and secondary antibodies, and visualized using an Odyssey infrared imager (LI-COR Biosciences).

Synergy Calculations and Statistical Methods

Drug combinations were tested in 3 to 7 independent repeat cell viability assays unless otherwise indicated Inhibition data were fit to a four-parameter logistic equation using GraphPad Prism. Potential non-additive effects were evaluated by the Bliss independence method (25-27). Briefly, the predicted Bliss value for the combination (F_{c}) was calculated as F_{c}=Fₐ+F_{b}−(Fₐ×F_{b}), where Fₐ and F_{b} represent the observed fractional growth inhibitions caused by compounds A and B used alone. Bliss differences were calculated by subtracting the predicted value (F_{c}) from the experimentally observed inhibition. Mean Bliss differences were calculated for each point in a matrix of drug-drug combinations, converted to percentages, and assessed for statistical significance from the null value of zero using two-sided t-tests with a significance level of 0.05. Statistical evaluations were not performed in cases where the fractional growth inhibition exceeded unity, since such values fall outside the Bliss framework. In addition, Bliss differences of less than 5% were considered to be of limited biological relevance and were excluded from synergy considerations.

### REFERENCES

1. MacVicar G R, Hussain M H. Emerging therapies in metastatic castration-sensitive and castration-resistant prostate cancer. Curr Opin Oncol 2013; 25:252-60.
2. Rathkopf D, Scher H I. Androgen receptor antagonists in castration-resistant prostate cancer. Cancer J 2013; 19:43-9.
3. Ferraldeschi R, de Bono J. Agents that target androgen synthesis in castration-resistant prostate cancer. Cancer J 2013; 19:34-42.
4. Richards J, Lim A C, Hay C W, Taylor A E, Wingate A, Nowakowska K, et al. Interactions of abiraterone, eplerenone, and prednisolone with wild-type and mutant androgen receptor: a rationale for increasing abiraterone exposure or combining with MDV3100. Cancer Res 2012; 72:2176-82.
5. Soifer H S, Souleimanian N, Wu S, Voskresenskiy A M, Collak F K, Cinar B, et al. Direct regulation of androgen receptor activity by potent CYP17 inhibitors in prostate cancer cells. J Biol Chem 2012; 287:3777-87.
6. Carver B S, Chapinski C, Wongvipat J, Hieronymus H, Chen Y, Chandarlapaty S, et al. Reciprocal feedback regulation of PI3K and androgen receptor signaling in PTEN-deficient prostate cancer. Cancer Cell 2011; 19:575-86.
7. Thomas C, Lamoureux F, Crafter C, Davies B R, Beralidi E, Fazli L, et al. Synergistic targeting of PI3K/AKT-pathway and androgen-receptor axis significantly delays castration-resistant prostate cancer progression in vivo. Mol Cancer Ther 2013; Epub ahead of print.
8. Wright G L, Jr., Grob B M, Haley C, Grossman K, Newhall K, Petrylak D, et al. Upregulation of prostate-specific membrane antigen after androgen-deprivation therapy. Urology 1996; 48:326-34.
9. Mostaghel E A, Page S T, Lin D W, Fazli L, Coleman I M, True L D, et al. Intraprostatic androgens and androgen-regulated gene expression persist after testosterone suppression: therapeutic implications for castration-resistant prostate cancer. Cancer Res 2007; 67:5033-41.
10. Evans M J, Smith-Jones P M, Wongvipat J, Navarro V, Kim S, Bander N H, et al. Noninvasive measurement of androgen receptor signaling with a positron-emitting radiopharmaceutical that targets prostate-specific membrane antigen. Proc Natl Acad Sci USA 2011; 108:9578-82.
11. Miyamoto D T, Lee R J, Stott S L, Ting D T, Wittner B S, Ulman M, et al. Androgen receptor signaling in circulating tumor cells as a marker of hormonally responsive prostate cancer. Cancer Discov 2012; 2:995-1003.
12. Foss C A, Mease R C, Cho S Y, Kim H J, Pomper M G. GCPII imaging and cancer. Curr Med Chem 2012; 19:1346-59.
13. Olson W C, Heston W D W, Rajasekaran A K. Clinical trials of cancer therapies targeting prostate-specific membrane antigen. Reviews on Recent Clinical Trials 2007; 2:182-90.
14. Perner S, Hofer M D, Kim R, Shah R B, Li H, Moller P, et al. Prostate-specific membrane antigen expression as a predictor of prostate cancer progression. Hum Pathol 2007; 38:696-701.
15. Ross J S, Sheehan C E, Fisher H A, Kaufman R P, Jr., Kaur P, Gray K, et al. Correlation of primary tumor prostate-specific membrane antigen expression with disease recurrence in prostate cancer. Clin Cancer Res 2003; 9:6357-62.
16. Silver D A, Pellicer I, Fair W R, Heston W D, Cordon-Cardo C. Prostate-specific membrane antigen expression in normal and malignant human tissues. Clin Cancer Res 1997; 3:81-5.
17. Chang S S, Reuter V E, Heston W D, Bander N H, Grauer L S, Gaudin P B. Five different anti-prostate-specific membrane antigen (PSMA) antibodies confirm PSMA expression in tumor-associated neovasculature. Cancer Res 1999; 59:3192-8.
18. Younes A, Bartlett N L, Leonard J P, Kennedy D A, Lynch C M, Sievers E L, et al. Brentuximab vedotin (SGN-35) for relapsed CD30-positive lymphomas. N Engl J Med 2010; 363:1812-21.
19. Verma S, Miles D, Gianni L, Krop I E, Welslau M, Baselga J, et al. Trastuzumab emtansine for HER2-positive advanced breast cancer. N Engl J Med 2012; 367:1783-91.
20. Ma D, Hopf C, Malewicz A D, Donovan G D, Senter P D, Goeckeler W F, et al. Potent antitumor activity of an auristatin-conjugated, fully human monoclonal antibody to prostate-specific membrane antigen. Clin Cancer Res 2006; 12:2591-6.
21. Wang X, Ma D, Olson W C, Heston W D. In vitro and in vivo responses of advanced prostate tumors to PSMA ADC, an auristatin-conjugated antibody to prostate-specific membrane antigen. Mol Cancer Ther 2011; 10:1728-39.
22. Wu H C, Hsieh J T, Gleave M E, Brown N M, Pathak S, Chung L W. Derivation of androgen-independent human LNCaP prostatic cancer cell sublines: role of bone stromal cells. Int J Cancer 1994; 57:406-12.
23. Veldscholte J, Berrevoets C A, Ris-Stalpers C, Kuiper G G, Jenster G, Trapman J, et al. The androgen receptor in LNCaP cells contains a mutation in the ligand binding domain which affects steroid binding characteristics and response to antiandrogens. J Steroid Biochem Mol Biol 1992; 41:665-9.
24. McMenamin M E, Soung P, Perera S, Kaplan I, Loda M, Sellers W R. Loss of PTEN expression in paraffin-embedded primary prostate cancer correlates with high Gleason score and advanced stage. Cancer Res 1999; 59:4291-6.
25. Bliss C I. The toxicity of poisons applied jointly. Ann Appl Biol 1939; 26:585-615.
26. Wallin J J, Guan J, Prior W W, Lee L B, Berry L, Belmont L D, et al. GDC-0941, a novel class I selective PI3K inhibitor, enhances the efficacy of docetaxel in human breast cancer models by increasing cell death in vitro and in vivo. Clin Cancer Res 2012; 18:3901-11.
27. Wong M, Tan N, Zha J, Peale F V, Yue P, Fairbrother W J, et al. Navitoclax (ABT-263) reduces Bcl-x(L)-mediated chemoresistance in ovarian cancer models. Mol Cancer Ther 2012; 11:1026-35.
28. Squillace R M, Miller D, Wardwell S D, Wang F, Clackson T, Rivera V M. Synergistic activity of the mTOR inhibitor ridaforolimus and the antiandrogen bicalutamide in prostate cancer models. Int J Oncol 2012; 41:425-32.
29. Wang Y, Mikhailova M, Bose S, Pan C X, deVere White R W, Ghosh P M. Regulation of androgen receptor transcriptional activity by rapamycin in prostate cancer cell proliferation and survival. Oncogene 2008; 27:7106-17.
30. Carracedo A, Ma L, Teruya-Feldstein J, Rojo F, Salmena L, Alimonti A, et al Inhibition of mTORC1 leads to MAPK pathway activation through a PI3K-dependent feedback loop in human cancer. J Clin Invest 2008; 118:3065-74.
31. O'Reilly K E, Rojo F, She Q B, Solit D, Mills G B, Smith D, et al. mTOR inhibition induces upstream receptor tyrosine kinase signaling and activates Akt. Cancer Res 2006; 66:1500-8.
32. Sarbassov D D, Guertin D A, Ali S M, Sabatini D M. Phosphorylation and regulation of Akt/PKB by the rictor-mTOR complex. Science 2005; 307:1098-101.
33. Easton J B, Kurmasheva R T, Houghton P J. IRS-1: auditing the effectiveness of mTOR inhibitors. Cancer Cell 2006; 9:153-5.
34. Hirai H, Sootome H, Nakatsuru Y, Miyama K, Taguchi S, Tsujioka K, et al. MK-2206, an allosteric Akt inhibitor, enhances antitumor efficacy by standard chemotherapeutic agents or molecular targeted drugs in vitro and in vivo. Mol Cancer Ther 2010; 9:1956-67.
35. Folkes A J, Ahmadi K, Alderton W K, Alix S, Baker S J, Box G, et al. The identification of 2-(1H-indazol-4-yl)-6-(4-methanesulfonyl-piperazin-1-ylmethyl)-4-morpholin-4-yl-t hieno[3,2-d]pyrimidine (GDC-0941) as a potent, selective, orally bioavailable inhibitor of class I PI3 kinase for the treatment of cancer. J Med Chem 2008; 51:5522-32.
36. Good J A, Wang F, Rath 0, Kaan H Y, Talapatra S K, Podgorski D, et al. Optimized S-trityl-L-cysteine-based inhibitors of kinesin spindle protein with potent in vivo antitumor activity in lung cancer xenograft models. J Med Chem 2013; 56:1878-93.
37. Cerami E, Gao J, Dogrusoz U, Gross B E, Sumer S O, Aksoy B A, et al. The cBio cancer genomics portal: an open platform for exploring multidimensional cancer genomics data. Cancer Discov 2012; 2:401-4.
38. Zhu M L, Horbinski C M, Garzotto M, Qian D Z, Beer T M, Kyprianou N. Tubulin-targeting chemotherapy impairs androgen receptor activity in prostate cancer. Cancer Res 2010; 70:7992-8002.
39. Darshan M S, Loftus M S, Thadani-Mulero M, Levy B P, Escuin D, Zhou X K, et al. Taxane-induced blockade to nuclear accumulation of the androgen receptor predicts clinical responses in metastatic prostate cancer. Cancer Res 2011; 71:6019-29.
40. Dang Z C, Lowik C W. Removal of serum factors by charcoal treatment promotes adipogenesis via a MAPK-dependent pathway. Mol Cell Biochem 2005; 268:159-67.
41. Tran C, Ouk S, Clegg N J, Chen Y, Watson P A, Arora V, et al. Development of a second-generation antiandrogen for treatment of advanced prostate cancer. Science 2009; 324:787-90.
42. Clegg N J, Wongvipat J, Joseph J D, Tran C, Ouk S, Dilhas A, et al. ARN-509: a novel antiandrogen for prostate cancer treatment. Cancer Res 2012; 72:1494-503.
43. Joseph J D, Lu N, Qian J, Sensintaffar J, Shao G, Brigham D, et al. A clinically relevant androgen receptor mutation confers resistance to second-generation antiandrogens enzalutamide and ARN-509. Cancer Discov 2013; 3:1020-9.
44. Chen C D, Welsbie D S, Tran C, Baek S H, Chen R, Vessella R, et al. Molecular determinants of resistance to antiandrogen therapy. Nat Med 2004; 10:33-9.
45. Pienta K J, Abate-Shen C, Agus D B, Attar R M, Chung L W, Greenberg N M, et al. The current state of preclinical prostate cancer animal models. Prostate 2008; 68:629-39.
46. Garcia J A, Danielpour D. Mammalian target of rapamycin inhibition as a therapeutic strategy in the management of urologic malignancies. Mol Cancer Ther 2008; 7:1347-54.
47. Sarker D, Reid A H, Yap T A, de Bono J S. Targeting the PI3K/AKT pathway for the treatment of prostate cancer. Clin Cancer Res 2009; 15:4799-805.
48. Bitting R L, Armstrong A J. Targeting the PI3K/Akt/mTOR pathway in castration-resistant prostate cancer. Endocr Relat Cancer 2013; 20:R83-R99.
49. Zhao L, Au J L, Wientjes M G. Comparison of methods for evaluating drug-drug interaction. Front Biosci (Elite Ed) 2010; 2:241-9.

What is claimed is:

1. A method of inhibiting proliferation of prostate-specific membrane antigen (PSMA)-expressing cancer cells, the method comprising:
contacting PSMA-expressing cancer cells with an antiandrogen, an mTOR inhibitor or prednisone; and
contacting the PSMA-expressing cancer cells with a PSMA ligand-anticancer agent conjugate or a PSMA ligand-cytotoxic agent conjugate.

2. The method of claim 1, wherein the PSMA ligand of the conjugate comprises an antibody, or antigen-binding fragment thereof, that binds specifically PSMA.

3-11. (canceled)

12. The method of claim 1, wherein the PSMA ligand of the conjugate comprises a small molecule ligand that binds specifically PSMA.

13-15. (canceled)

16. The method of claim 1, wherein the anticancer agent comprises an auristatin, tubulysin, a pyrrolobenzodiazepine dimer, calicheamicin, colchicine, ispinesib, combrestatin A4, maytansinoid DM1, maytansinoid DM4, doxorubicin, or a cytotoxic radionuclide.

17-18. (canceled)

19. The method of claim 1, wherein the PSMA ligand-anticancer agent conjugate comprises BIND-014.

20. The method of claim 1, wherein the compound that increases cell surface expression of PSMA is an antiandrogen.

21-23. (canceled)

24. The method of claim 1, wherein the antiandrogen is abiraterone, enzalutamide, nilutamide, flutamide, bicalutamide, ARN 509, galeterone or orteronel.

25-26. (canceled)

27. The method of claim 1, wherein the step of contacting the PSMA-expressing cancer cells with the antiandrogen and the step of contacting the PSMA-expressing cancer cells with the PSMA ligand-anticancer agent conjugate are concurrent.

28. The method of claim 1, wherein the step of contacting the PSMA-expressing cancer cells with the antiandrogen and the step of contacting the PSMA-expressing cancer cells with the PSMA ligand-anticancer agent conjugate are sequential.

29-30. (canceled)

31. The method of claim 1, wherein the PSMA-expressing cancer cells are in contact with the antiandrogen for at least 3 days.

32-34. (canceled)

35. The method of claim 1, wherein the PSMA-expressing cancer cells comprise androgen-independent PSMA-expressing cancer cells.

36. The method of claim 1, wherein the PSMA-expressing cancer cells comprise PSMA-expressing cancer cells insensitive to antiandrogen therapy.

37. (canceled)

38. The method of claim 1, wherein the PSMA-expressing cancer cells are of a tumor.

39. The method of claim 1, wherein the PSMA-expressing cancer cells are PSMA-expressing prostate cancer cells.

40. The method of claim 1, wherein the PSMA-expressing cancer cells are PSMA-expressing non-prostate cancer cells.

41. (canceled)

42. The method of claim 1, wherein the PSMA-expressing cancer cells are of a subject.

43-47. (canceled)

48. The method of claim 1, wherein the antiandrogen is enzalutamide, abiraterone or ARN 509, and the PSMA ligand-anticancer agent conjugate is a PSMA ADC.

49-87. (canceled)

88. A method of specific delivery of a cytotoxic agent to PSMA-expressing cells in a subject, comprising:
administering to a subject a compound that increases cell surface expression of PSMA; and
administering to the subject a PSMA ligand-cytotoxic agent conjugate.

89-136. (canceled)

137. A composition comprising (a) a compound that increases cell surface expression of PSMA and a PSMA ligand-anticancer agent conjugate or PSMA ligand-cytotoxic agent conjugate, or (b) prednisone and a PSMA ligand conjugate.

138-165. (canceled)

166. A kit comprising:
a container containing prednisone or a compound that increases cell surface expression of PSMA of a PSMA-expressing cell; and
a container containing a PSMA ligand conjugate or PSMA ligand-anticancer agent conjugate or PSMA ligand-cytotoxic agent conjugate.

167-245. (canceled)
